(19) United States
(12) Patent Application Publication
(10) Pub. No.: US 2023/0151077 A1
(43) Pub. Date: May 18, 2023

(54) SOLUBLE ACE2 FOR TREATMENT OF COVID-19

(71) Applicant: MASKER MED TECH AB, Malmö (SE)

(72) Inventors: György MARKO-VARGA, Malmö (SE); Balazs DÖME, Vienna (AT); Viktoria LASZLO, Budapest (HU); Peter DÖME, Budapest (HU); Yutaka SUGIHARA, Malmö (SE); Jeovanis GIL VALDES, Lund (SE); Roger APPELQVIST, Malmö (SE); Johan MALM, Malmö (SE)

(21) Appl. No.: 17/917,420

(22) PCT Filed: Apr. 9, 2021
(86) PCT No.: PCT/SE2021/050327

§ 371 Date: Oct. 6, 2022

(30) Foreign Application Priority Data
Apr. 9, 2020 (SE) 2050413-0
May 6, 2020 (SE) 2050521-0
Nov. 9, 2020 (SE) 2051305-7

(51) Int. Cl. C07K 14/72; A61P 31/14

(52) U.S. Cl. CPC C07K 14/72; A61P 31/14

(57) ABSTRACT

In the invention is provided a polypeptide comprising at least 75 amino acids and having an amino acid sequence having at least 90%, such as at least 95%, 96%, 97, 98%, or 99%, such as 100% sequence identity (% SI) with human Angiotensin-converting enzyme 2 (ACE2) (SEQ ID NO 1), for use in the treatment of COVID19, SARS, or MERS, characterized in that the polypeptide is administered pulmonary and/or nasally. Further is provided the polypeptide for use in reducing the number of active virus particles being exhaled by a treated subject infected by SARS-CoV-2, SARS-CoV, or Mers-CoV. Also a dry powder or an aerosol comprising the polypeptide.

### FIELD OF THE INVENTION

This invention pertains in general to the field of the use of a polypeptide for pulmonary and/or nasal treatment. More particularly the invention relates to the use of cell surface receptor angiotensin-converting enzyme 2 (ACE2), or domains thereof, for pulmonary and/or nasal treatment. More particularly, the invention relates to pulmonary and/or nasal administration for treatment of COVID-19. Furthermore, the present invention pertains to a dry powder or an aerosol comprising said polypeptide suitable for pulmonary treatment.

### BACKGROUND OF THE INVENTION

Viral disease COVID-19, caused by the β-type coronavirus SARS-CoV-2, has emerged in Wuhan (Hubei, China) in December 2019 and quickly became a global pandemic [1, 2]. Accordingly, as of 07. April 2020. more than 1.4 million people have infected with SARS-CoV-2 and 81103 death cases have occurred (https://www.arcgis.com/apps/opsdashboard/index.html #/bda7594740fd40299423467b48e9ecf6). Asymptomatic infections have been described, but their exact frequency is unknown [3]. The great majority of infected individuals (81%) shows only mild symptoms, but considerable proportions of them have severe (14%) or critical (5%) illness [3, 4]. Mortality data show great variance in different countries with a range from 0.9% (South Korea) to 7.2% (Italy) [3].

Currently, specific and effective drugs are unavailable and the mainstay of COVID-19 treatment is supportive care [1]. At the same time, enormous efforts are currently carried out globally to identify drugs that may be effective against SARS-CoV-2 infection. These approaches are typically based on the idea of drug repurposing (e.g. using the antimalarial drug chloroquine or antiviral agents [e.g. favipiravir] not designed directly against coronaviruses) [1-3]. In this wise, the need for any treatment approaches that are specifically targeted against coronaviruses is highly justifiable. Of note, our treatment approach utilizes the interaction between human ACE2 and the viral S protein and—as S protein is an ubiquiter component of coronaviruses—it may be used not only against SARS-CoV-2 but against any other coronaviruses too [5].

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a comprising at least 75 amino acids and having an amino acid sequence having at least 90%, such as at least 95%, 96%, 97, 98%, or 99%, such as 100% sequence identity (% SI) with human Angiotensin-converting enzyme 2 (ACE2) (SEQ ID NO 1), for use in the treatment of COVID19, SARS, or MERS, characterized in that the polypeptide is administered pulmonary and/or nasally.

Furthermore, is provided a polypeptide comprising at least 75 amino acids and having an amino acid sequence with at least 90%, such as at least 95%, 96%, 97, 98%, or 99%, such as 100% sequence identity (% SI) with human ACE2 (SEQ ID NO 1) for use in reducing the number of active virus particles being exhaled by a treated subject infected by SARS-CoV-2, SARS-CoV, or Mers-CoV.

Also a dry powder or an aerosol comprising a polypeptide comprising at least 75 amino acids and having an amino acid sequence having at least 90 such as at least 95%, 96%, 97, 98%, or 99%, such as 100% sequence identity (% SI) with human Angiotensin-converting enzyme 2 (SEQ ID NO 1).

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1. shows the mechanism of SARS-CoV-2 entry into the cells of the bronchial/alveolar wall and proposed therapeutic intervention. FIG. 1.A. SARS-CoV-2 infection is mediated by the interaction of the viral spike (S) protein and its functional receptor, ACE2. The plasma membrane forms an endosome around the virus and the virus enters cells by endocytosis. FIG. 1.B. Inhalation of soluble ACE2 saturates the available S proteins, blocks the abovementioned interaction and abrogates infection,

FIG. 2 shows the sequence coverage confirmation of peptide 2MA1 (SEQ ID NO 2)-main band, by mass spectrometry, the bands representing the fragments sequenced;

FIG. 3 shows an MS/MS spectrum of the double charged signal with 517.26 as the molecular mass (The spectrum was matched with the theoretical sequence of the N-terminal peptide of 2MA1 (SEQ ID NO 2);

FIG. 4 shows MS/MS spectra of the triple charged signal 778.74 Th corresponding to the peptide from 2MA1 (SEQ ID NO 2) 58-77 (A) and the double charged signal 649.32 Th from the peptide 325-336 (B);

FIG. 5 shows gel results for the stability of peptide 2MA1 (SEQ ID NO 2) which was investigated with perspective to pH-window and sample composition;

FIG. 6 shows gel results for the stability of ACE2 that was investigated by different buffer conditions (with pH 5 and 11);

FIG. 7 shows a plot of the binding properties of peptide 2MA1 (SEQ ID NO 2), to the SPIKE protein of SARS COVID-2;

FIG. 8 shows a plot of the peptide 2MA1 (SEQ ID NO 2) binding kinetics to RBD (receptor binding domain) of the SPIKE protein;

FIG. 9 shows a plot of de-glycosylated peptide 2MA1 (SEQ ID NO 2) binding kinetics to RBD of the SPIKE protein;

FIG. 10 shows gel results for binding of ECD (extra cellular domain of spike protein) to E. coli expressed ACE2 extracellular domain after 10 minutes incubation time;

FIG. 11 shows gel results for binding of ECD to E. coli expressed ACE2 extracellular domain after 10 hours incubation time;

FIG. 12 shows mesh vaporization of the peptide (here according to SEQ ID NO 2) of the invention;

FIG. 13 shows jet vaporization of the peptide (here according to SEQ ID NO 2) of the invention;

FIG. 14 shows intrapulmonary levels of ACE2 (2MA1) determined by Western blot analysis after injecting 1 μg protein/mouse;

FIG. 15 shows intrapulmonary levels of ACE2 (2MA1) determined by Western blot analysis after injecting 5 μg protein/mouse;

FIG. 16. shows plasma levels of ACE2 (2MA1) after intrapulmonary delivery of the protein;

FIG. 17. shows representative histology images of the lungs of control mice 30 min/6 h/24 h/48 h after injection;

FIG. 18. shows representative histology images of the lungs of mice that received 1 μg ACE2 (2MA1) 30 min/6 h/24 h/48 h after injection;

FIG. 19. shows representative histology images of the lungs of mice that received 5 μg ACE2 (2MA1) 30 min/6 h/24 h/48 h after injection;

FIG. 20 shows the RBD (here SARS-CoV-2 Spike 51 Receptor Binding Domain Protein) and 2MA1 kinetics in mouse lung;

FIG. 21 shows gel results for filtration procedure for, wherein the first two columns show that free RBD (here SARS-CoV-2 Spike 51 Receptor Binding Domain Protein) go into the flow-through fraction, while the second two columns shows that the RBD-ACE2 protein complex stayed in the filtration device;

FIG. 22 shows identified 2MA1 protein sequence by MS analysis in mouse lung experiments;

FIG. 23 shows identified RBD (here SARS-CoV-2 Spike 51 Receptor Binding Domain Protein) protein sequence by MS analysis in mouse lung experiments;

FIG. 24 shows mass spectra correctly assigned to peptide sequences from 2MA1 marked in FIG. 22;

FIG. 25 shows mass spectra correctly assigned to peptide sequences from 2MA1 marked in FIG. 22;

FIG. 26 shows mass spectra correctly assigned to peptide sequences from RBD protein sequence marked in FIG. 23;

FIG. 27 shows mass spectra correctly assigned to peptide sequences from RBD protein sequence marked in FIG. 23;

FIG. 28 shows MS/MS spectra with a comparison of signals between the supernatant (top) and the flow-through (bottom) in mouse lung experiments;

FIG. 29 shows MS/MS spectra with a comparison of signals between the supernatant (top) and the flow-through (bottom) in mouse lung experiments; and

FIG. 30 shows gel results for the stability and binding ability (pulldown) of dry powder form peptide 2MA1 (SEQ ID NO 2) being re-dissolved after 48 hour storage, compared to pulled down samples with and without 2 μg of ACE2 (2MA1) in solution.

### DESCRIPTION OF EMBODIMENTS

The following description focuses on an embodiment of the present invention applicable to a polypeptide comprising part of the sequence of cell surface receptor angiotensin-converting enzyme 2 (ACE2), suitable for pulmonary treatment.

Although ACE2 is expressed in various organs (e.g. the kidneys and the gastrointestinal tract), type 2 pneumocytes express high amounts of ACE2 [6]. The extracellular domain of the full-length ACE2 is anchored to the plasma membrane by its transmembrane domain. Importantly, both SARS-CoV [7] and SARS-CoV-2 [1] utilize ACE2 as a receptor to enter the target cell, but the SARS-CoV-2 binds ACE2 with higher affinity than SARS-CoV [8]. It has been speculated that the soluble form of ACE2 may compete with the membrane-bound form and thus inhibiting viral infection. Indeed, ACE2 expression on different cell lines correlates with susceptibility to SARS-CoV infection [9]. Replication of SARS-CoV could effectively be blocked by soluble ACE2 in monkey kidney cells and, moreover, while SARS-CoV could not infect 293T cells lacking ACE2, they effectively replicated in ACE2 transfected cells [10]. The extracellular domain of ACE2 conjugated to the Fc region of the IgG1 potently neutralized SARS-CoV and SARS-CoV-2 in vitro.

In a very recent report, clinical-grade recombinant human (rh) soluble ACE2 (hrsACE2) has been shown to significantly reduce SARS-CoV-2 viral growth in vitro [11]. In line with this, a randomized, double-blind Phase II trial has been started to evaluate the efficacy and safety of the intravenously (i.v.) given recombinant form of ACE2 (APN01) in severely infected COVID-19 patients in Europe [12]. Surprisingly, despite that the respiratory tract is being the primary COVID19 target and that there is enormous international interest in using ACE2 to treat COVID19 patients, no attempts have been reported so far to study the efficacy of inhaled ACE2-Proteoforms.

Thus, in the invention we find that the optimum route of administration of ACE2, or a peptide according to the invention, is pulmonary inhalation. Not only does the lung offer a large surface area for drug absorption, the ACE2, or peptide of the invention, also acts as a neutralizer for virus particles both in the upper and lower respiratory tract, and polypeptides and deactivated virus particles may both be expelled as phlegm, making administration safe for the COVID-19 patients.

When referring to ACE2 (for inhalation) below, it is understood that this refers to human ACE2 protein, or a peptide of the invention.

Once locally administered, SARS-CoV-2 virus particles will bind to the ACE2 proteins, either the administered (exogenous) ACE2, or on the host RCE2. Published surface plasmon resonance experiments probing the binding kinetics for human ACE2 and immobilized 2019-nCoV shows that SARS-CoV-2 S protein binds to the PD of ACE2 at high affinity (a dissociation constant (Kd) of ˜15 nM) [8]. As such, the administered ACE2, or peptide of the invention, will serve to occupy active virus particles, by competitive binding, thus neutralizing part of the virus particles.

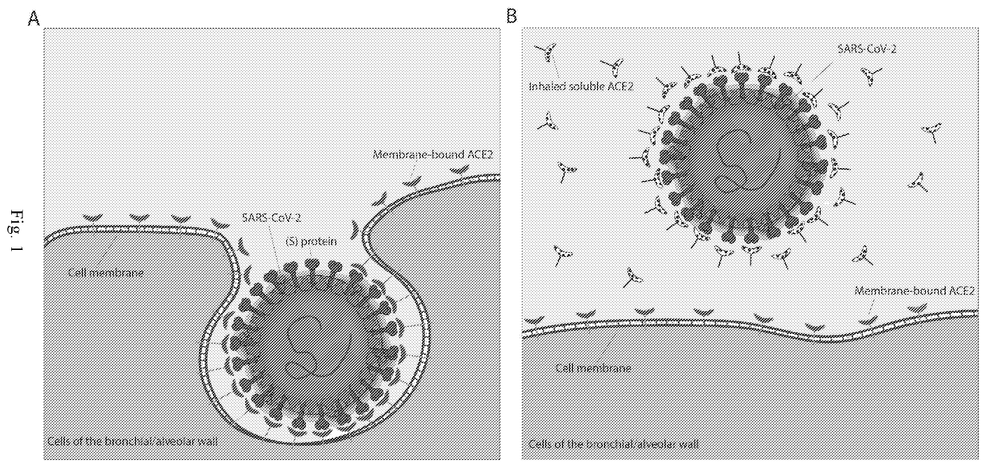

By introducing inhaled rhACE2, or a peptide according to the invention, into the COVID19-infected respiratory tract, a competitive action will take place with a dynamic equilibrium that will determine the affinity and binding kinetics of the virus particles for their receptors (i.e. host ACE2 vs. exogenous rhACE2). With a given dosing, the kinetic rate constants and equilibrium constants will favour the COVID19-rhACE2 complex formation. Similarly, by increasing the dose, the equilibrium can be pushed further towards COVID19-rhACE2 complex formation. This is effectually illustrated by FIG. 1, which shows an example of such virus particle neutralization.

In contrast to i.v. administration, inhalation is a promising non-invasive method of rhACE2 delivery to treat COVID19 patients, as it will result high drug levels in the lung, while, depending on drug formulation, limiting rhACE2 passage into the pulmonary capillaries (i.e. the circulation). Importantly, inhaled ACE2 will also avoid any first-pass metabolism in the liver.

The most important advantage of inhaled (vs. intravenously given) ACE2 would be, therefore, that, as viruses attack the lung (the mainly damaged organ in COVID-19) from the alveolar space, their deactivation would be carrying out on the spot. In addition, as ACE2 is a key enzyme of the renin-angiotensin system with various biological activities and it is expressed in at least 15 organs [13], we may expect that administering ACE2 via inhalation (v.s. given i.v.) will result lower serum levels and, consequently, less severe systemic side effects

Importantly, inhaled ACE2 could also be used in COVID19 patients with less severe symptoms to reduce the number of virus particles in their exhaled breath and in this wise reduce their capability to infect other subjects.

Early data from COVID-19 patients suggest that a large amount of virus particles is present in patients' nasal cavities, possibly before they have symptoms and likely in the first week of the disease [14]. As such, it is advantageous to use the peptide of the invention such that the peptide passes through the nasal cavity of the patient. This may happen automatically during pulmonary administration, for instance when administering an aerosol using a mask covering both mouth and nose of the patient. However, the nasal cavity may also be reached using simpler targeted administration, such as using a nose spray comprising the peptide of the invention, or by administering the peptide of the invention using any other suitable intranasal drug delivery system.

Human ACE2 has the sequence according to Uniprot reference Q9BYFI (ACE2_HUMAN Angiotensin-converting enzyme), provided as SEQ ID NO 1, as shown in table 1.

TABLE 1
SEQUENCE LIST

SEQ ID NO 1:
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV
EKAIRMSRSRINDAFRLNDNSLEFLGIQPTLGPPNQPPVSIWLIVFGVVM
GVIVVGIVILIFTGIRDRKKKNKARSGENPYASIDISKGENNPGFQNTDD
VQTSF
SEQ ID NO 2:
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS
SEQ ID NO 3:
QSTIEEQAKTFLDKFNHEAEDLFYQSSLASWNYNTNITEENVQNMNNAGD
KWSAFLKEQSTLAQMYPLQEIQDAKIKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS
SEQ ID NO 4:
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEM
SEQ ID NO 5:
QSTIEEQAKTFLDKFNHEAEDLFYQSSLASWNYNTNITEENVQNMNNAGD
KWSAFLKEQSTLAQMYPLQEIQDAKIKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEM
SEQ ID NO 6:
QSTIEEQAKTFLDKFNHEAEDLFYQSSLASWNYNTNITEENVQNMNNAGD
KWSAFLKEQSTLAQMYPLQEIQNLTVKLQLQALQQN
SEQ ID NO 7:
QSTIEEQAKTFLDKFNHEAEDLFYQSSLASWNYNTNITEENVQNMNNAGD
KWSAFLKEQSTLAQMYPLQEIQDAKIKLQLQALQQN

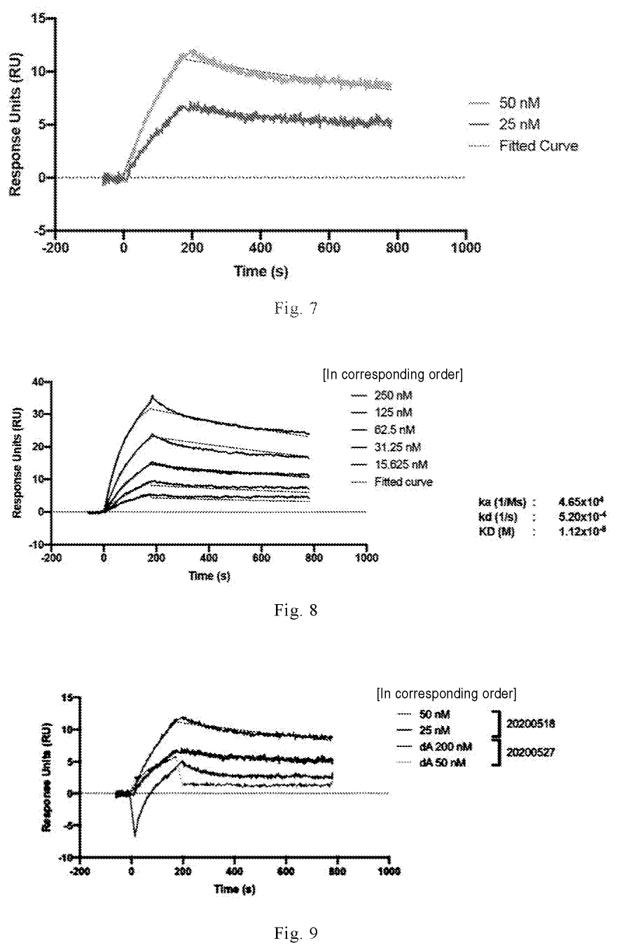

Importantly, SARS-CoV-2 spike protein does not bind to all of ACE2. The spike protein only interacts with part of the extracellular domain of the ACE2. The extracellular domain of ACE2 is amino acids 18 to 740 of SEQ ID NO 1 (1 to 17 is the signal peptide that might be removed upon activation, 741 to 761 is the transmembrane domain and 762 to 805 the cytoplasmic domain), which is shown as SEQ ID NO 2 in table 1. Binding of the peptide according to SEQ ID NO 2 of the invention can be seen in FIG. 8.

Cryo-electron microscopy structural studies suggests that the binding is between the ectodomain part of SARS-CoV-2 S protein which binds to the ACE2 N-terminal peptidase domain (PD), which is amino acids 19 to 615 of SEQ ID NO 1 [15]. Also, binding has been shown even if there are smaller sequence variations of ACE2. As such, a polypeptide comprising part of the ACE2 sequence is enough to neutralize the virus particles by binding.

Further, in table 1, SEQ ID 4 shows the extracellular subdomain I. This domain contains all reported interaction points with the spike-protein, however, the subdomain does not retain the ACE2 activity. This may be of advantage, since the peptide will not have several parallel effects, for instance making it easier to evaluate treatment results.

SEQ ID NO 6 shows the partial extracellular subdomain I, which is a self-containing domain section comprising two out of three reported interaction points with the spike protein. As such, the partial subdomain will retain its structure to interact with the spike protein, while not having ACE2 enzymatic activity and while having reduced size and a less complicated protein folding motif than the whole ACE2 protein.

It has been shown that a mutation that altered the NxT/S motif in humanACE2 to a civet ACE2-like sequence (90-NLTV-93 to DAKI), expected to abolish the N-glycosylation, increased the SARS-CoV infectivity and S-protein binding (https://doi.org/10.1101/2020.04.07.024752), [16, 17]. As such, SEQ ID 3, SEQ ID 5, and SEQ ID 7 represents the sequences of SEQ ID 2, SEQ ID 3, 4 and SEQ ID 6, respectively, with this mutation. As such, it is likely that these peptides will retain higher binding affinity for the spike protein.

COVID-19, severe acute respiratory syndrome (SARS) and Middle East respiratory syndrome (MERS) are all caused by related coronaviruses (SARS-CoV-2, SARS-CoV, and Mers-CoV, respectably). Some people infected with MERS coronavirus (MERS-CoV) develop severe acute respiratory illness, including fever, cough, and shortness of breath. Infection with SARS coronavirus (SARS-CoV) can cause a severe viral respiratory illness. As such, the polypeptide of the invention can likely be used in the treatment of COVID19, SARS, and MERS.

In one embodiment, is provided a polypeptide comprising at least 75 amino acids and having an amino acid sequence having at least 90%, such as at least 95%, 96%, 97, 98%, or 99%, such as 100%, sequence identity (% SI) with human Angiotensin-converting enzyme 2 (SEQ ID NO 1), for use in the treatment of COVID19, SARS, or MERS, characterized in that the polypeptide is administered pulmonary and/or nasally. In one further embodiment, the polypeptide is administered pulmonary and nasally. In one further embodiment, the polypeptide is administered pulmonary. In one further embodiment, the polypeptide is administered nasally. In one further embodiment, the polypeptide is for use in the treatment of COVID19. In one embodiment, the polypeptide comprises at least 76 amino acids, such as at least 77, 78, 79, 80, 81, 82, 83, or 84 amino acids, and shares at least 90%, such as at least 95%, 96%, 97, 98%, or 99%, such as 100% sequence identity (% SI) with SEQ ID 2 or SEQ ID NO 3.

In one embodiment, the polypeptide comprises at least 400 amino acids, such as at least 405, 406, 407, 408, 409, 410, 411, or 412 amino acids, and shares at least 90%, such as at least 95%, 96%, 97, 98%, or 99%, such as 100% sequence identity (% SI) with SEQ ID 4 or SEQ ID NO 5.

In one embodiment, the polypeptide comprises at least 500 amino acids and shares at least 90%, such as at least 95%, 96%, 97, 98%, or 99%, such as 100% sequence identity (% SI) with SEQ ID 1.

In one embodiment, the polypeptide comprises at least 500 amino acids and having an amino acid sequence having at least 90% sequence identity (% SI) with human Angiotensin-converting enzyme 2 (SEQ ID NO 1), for use in the treatment of COVID19, SARS, or MERS, characterized in that the polypeptide is administered pulmonary.

In one embodiment, the peptide comprises at least 700 amino acids, such as at least 710, 715, 716, 717, 718, 719, 720, 721, or 722 amino acids, and shares at least 90%, such as at least 95%, 96%, 97, 98%, or 99%, such as 100% sequence identity (% SI) with SEQ ID 6 or SEQ ID NO 7.

In one embodiment, the polypeptide comprises at least 90% sequence identity to amino acid residues 18 to 740 of SEQ ID NO 1.

In one embodiment, the polypeptide comprises at least 90% sequence identity to amino acid residues 19 to 615 of SEQ ID NO 1.

In one embodiment, the polypeptide is of at least 510, 520, 530, 540, 550, 560, 570, 580, 590, 591, 592, 593, 594, 595, 596 amino acids length.

In one embodiment, the polypeptide is of at least 645, 655, 665, 675, 685, 695, 705, 715, 716, 717, 718, 719, 720, 721, 722 amino acids length.

In one embodiment, the polypeptide is of at least 760, 765, 770, 775, 780, 785, 786, 787, 788, 790, 795, 800, 801, 802, 803, 804, 805 amino acids length.

In one embodiment, the polypeptide comprises at least 90%, 91%, 92%, 93%, 94%, 95%, 96%, 97%, 98%, 99% or 100% sequence identity to SEQ ID NO 1 or amino acid residues 18 to 740 of SEQ ID NO 1 or amino acid residues 19 to 615 of SEQ ID NO 1.

In one embodiment, the polypeptide shares at least 90%, such as at least 95%, 96%, 97, 98%, or 99%, such as 100% sequence identity (% SI) with the extracellular domain of human ACE2.

Sequence identity (% SI) as described herein may be assessed by any convenient method. Programs that compare and align pairs of sequences, like ALIGN [18], FASTA [19] and gapped BLAST [20], or BLASTP [21] can be used for this purpose. If no such resources are at hand, according to one embodiment, sequence identity (% SI) can be calculated as (% SI)=100%*(Nr of identical residues in pairwise alignment)/(Length of the shortest sequence).

In one embodiment, the polypeptide is the ACE2-PD domain. In one further embodiment, the polypeptide is the extracellular domain of ACE2. In one further embodiment, the polypeptide is the ACE2 protein.

In one embodiment, the peptide shares at least 90%, such as at least 95%, 96%, 97, 98%, or 99%, such as 100% sequence identity (% SI) to the Angiotensin-converting enzyme 2 (ACE2) N-terminal peptidase domain (PD) domain. In one further embodiment, the sequence of the ACE2 PD domain is amino acids 19 to 615 of SEQ ID NO 1.

In one embodiment, the peptide shares at least 90%, such as at least 95%, 96%, 97, 98%, or 99%, such as 100% sequence identity (% SI) to the ACE2 extracellular domain. In one further embodiment, the sequence of the extracellular domain of ACE2 is amino acids 18 to 740 of SEQ ID NO 1.

In one embodiment, the peptide shares at least 95% sequence identity (% SI) to ACE2. In one further embodiment, the ACE2 is human ACE2.

In one embodiment, the polypeptide is the ACE2-PD domain. In one further embodiment, the polypeptide is the extracellular domain of ACE2. In one further embodiment, the polypeptide is the ACE2 protein. In one further embodiment, the ACE2 is human ACE2.

However, protein molecules arising from all combinatorial sources of variation giving rise to products arising from a single gene. These include products differing due to genetic variation, alternatively spliced RNA, transcripts, and post-translational modifications. As such, the polypeptide may be the proteoform of ACE2 human gene (ORF Names:UNQ868/PRO1885).

Thus, in one embodiment, the polypeptide is the proteoform of the human ACE2 gene.

Post-translational modifications, such as glycosylation, may also affect the SARS-CoV-2 spike protein to ACE2 interaction. Possibly, such modifications may serve to activate the protein and enhance binding.

In one embodiment, the peptide has post-translational modifications, such as glycosylation.

The known Post-translational modifications of ACE2 are glycosylation on positions 53, 90, 103, 322, 432, 546 and 690 and disulphide bridges between positions 133-141, 344-361, 530-542.

In one further embodiment, the post-translational modifications are glycosylations of positions 53, 90, 103, 322, 432, 546 and 690.

As mentioned, inhaled ACE2, or a polypeptide of the invention, could also be used in COVID19 patients with less severe symptoms to reduce the number of virus particles in their exhaled breath and in this wise reduce their capability to infect other subjects.

In one embodiment, the treatment of COVID19 reduces the number of active virus particles being exhaled by a treated subject.

In one embodiment, is provided a polypeptide comprising at least 75 amino acids and having an amino acid sequence with at least 90%, such as at least 95%, 96%, 97, 98%, or 99%, such as 100% sequence identity (% SI) with human ACE2 (SEQ ID NO 1) for use in reducing the number of active virus particles being exhaled by a treated subject infected by SARS-CoV-2, SARS-CoV, or Mers-CoV. In one further embodiment, wherein the subject is infected by SARS-CoV-2. In one further embodiment, is provided a polypeptide comprising at least 500 amino acids and having an amino acid sequence with at least 90% sequence identity (% SI) with human ACE2 (SEQ ID NO 1).

In one embodiment, the polypeptide comprises at least 76 amino acids, such as at least 77, 78, 79, 80, 81, 82, 83, or 84 amino acids, and shares at least 90%, such as at least 95%, 96%, 97, 98%, or 99%, such as 100% sequence identity (% SI) with SEQ ID 2 or SEQ ID NO 3.

In one embodiment, the polypeptide comprises at least 400 amino acids, such as at least 405, 406, 407, 408, 409, 410, 411, or 412 amino acids, and shares at least 90%, such as at least 95%, 96%, 97, 98%, or 99%, such as 100% sequence identity (% SI) with SEQ ID 4 or SEQ ID NO 5.

In one embodiment, the polypeptide comprises at least 500 amino acids and having an amino acid sequence with at least 90% sequence identity (% SI) with human ACE2 (SEQ ID NO 1).

In one embodiment, the peptide comprises at least 700 amino acids, such as at least 710, 715, 716, 717, 718, 719, 720, 721, or 722 amino acids, and shares at least 90%, such as at least 95%, 96%, 97, 98%, or 99%, such as 100% sequence identity (% SI) with SEQ ID 6 or SEQ ID NO 7.

In one embodiment, the polypeptide comprises at least 90%, such as at least 95%, 96%, 97, 98%, or 99%, such as 100% sequence identity (% SI) with human ACE2 N-terminal peptidase domain (PD) domain (SEQ ID NO 1), wherein the sequence of the PD domain is amino acids 19-615 of SEQ ID NO 1.

In one embodiment, wherein the peptide shares at least 90%, such as at least 95%, 96%, 97, 98%, or 99%, such as 100% sequence identity (% SI) to the extracellular domain of ACE2, wherein the sequence of the extracellular domain is amino acids 18-740 of SEQ ID NO 1.

ACE2 is a potent negative regulator of the renin-angiotensin system (RAS), a type I transmembrane glycoprotein that belongs to the M2 family of zinc metallopeptidases. ACE2 is responsible for production of angiotensin-(1-7) (Ang 1-7) by cleaving Ang II [22].

Ang II has been described as a pro-inflammatory molecule by binding to its angiotensin II type 1 (AT1) receptors [23] by inducing the production of reactive oxygen species such as superoxide and hydrogen peroxide [24] and by activating nuclear factor (NF)-kB, a transcription factor regulating the expression of several inflammatory cytokines, such as TNFa, IL-6, IL-8. IL-12, nitric oxide (NO), monocyte chemotactic protein-1 and intracellular adhesion molecule-1 (ICAM-1), which triggers an increase in vascular permeability and facilitates development of pulmonary edema [25, 26].

ACE2 acts as an anti-inflammatory protein, by counteracting the actions of Ang II [27]. Its product, Ang-(1-7) acts as an AT1 receptor antagonist [28]. The anti-inflammatory effect of Ang-(1-7) can also be mediated by the Mas receptors [22]. The interaction of Ang-(1-7) and Mas receptors regulate the phosphoinositide 3-kinase (PI3K)/AKT and extracellular signal-regulated kinases (ERK) signaling pathways and downstream effectors such as NO, FOXO1 (forkhead box 01) and COX-2 (cyclo-oxygenase-2) [29].

The RAS is one of the central players in inflammatory diseases of the respiratory tract, such as acute respiratory distress syndrome (ARDS), which is the major cause of death in COVID-19 patients. ARDS is the most severe form of acute lung injury, characterized by the release of pro-inflammatory cytokines and, as a result, a strong inflammatory response. Through the abovementioned mechanisms, the cleavage of Ang II to Ang-(1-7) can improve inflammation in ARDS. Both Ang-(1-7) and ACE2 had protective effect in different preclinical models of acute lung injury [30, 31]. Ace2 knockout mice developed more severe acute lung injury, compared to wild type (wt) mice. Importantly, the administration of catalytically active recombinant ACE2 protein improved the symptoms of acute lung injury in both the Ace2 knockout and wt mice [31].

As such, if ACE2 activity is retained in the peptide of the invention, the anti-inflammatory effect may benefit the patient. Especially, it would benefit patients with severe symptoms, such as patients having ARDS.

In one embodiment, the peptide is active ACE2 and acts as an anti-inflammatory protein.

In one embodiment, the peptide has anti-inflammatory effect through ACE2 activity.

In one embodiment, the patient has more advanced stage of the infection or severe symptoms, such as acute respiratory distress syndrome (ARDS).

A similar strategy can be adopted for the shorter peptide of the invention, that does not retain ACE2 activity, by co-administration with an anti-inflammatory agent. Examples of such agents may be glucocorticoids and nonsteroidal anti-inflammatory drugs. Some anti-inflammatory agents may have very low dose, such as interferon alpha. Anti-inflammatory agents are sometimes administered together with immunosuppressants, such as azathioprine, cyclosporin A, or cyclophosphamide.

In one further embodiment, the peptide of the invention is co-administered with an anti-inflammatory agent and/or an immunosuppressant.

In one embodiment, the peptide of the invention is co-administered with an anti-inflammatory agent.

In one embodiment, the anti-inflammatory agent is a glucocorticoid and/or nonsteroidal anti-inflammatory drug. Another patient group that will benefit from a treatment using the peptide of the invention is patients that for some reason cannot be vaccinated, when a vaccine becomes available. This includes patients allergic to components (or trace components) of a vaccine. Furthermore, older patients, or patients with a defective immune system, may not obtain a good protection using a vaccine.

There is a worry that it is a risk connected to using a vaccine at the same time as one might be infected (such as during a virus outbreak), and as such patients already infected, or in high risk of being infected, might benefit from the treatment using the peptide of the invention.

The treatment will help the patient through the infection with less risk, and likely the patient will build up a natural immunity to the virus.

In one embodiment, the treatment is for patients that cannot use a vaccine. In one further embodiment, the treatment will reduce risk for the patient (i.e. increase survival chance) while the patient develops natural immunity.

In the invention is provided a dry powder or an aerosol comprising the polypeptide as descried above. Aerosol administration of pharmaceutical compositions has been previously reported in treating a number of disorders. For example, respiratory delivery of aerosolized insulin solutions has been described in substantial detail. Administration of peptides via inhalation has been shown to work. In this wise, the administration of nebulized ACE2 in regard to efficacy and safety prima facie seems to be plausible.

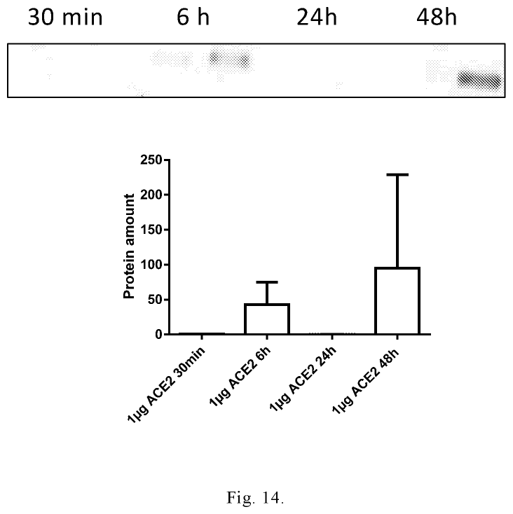

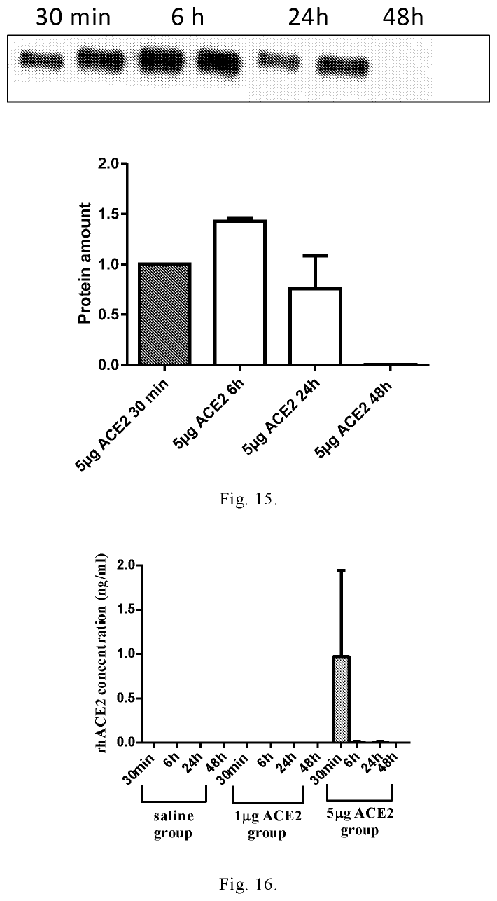

Mouse model experiments confirmed the intrapulmonary stability of ACE2 in an in vivo mouse model. As seen in FIG. 14, the peptide was barely detectable in the lungs of mice that received 1 μg ACE2 (2MA1), while it was present and very stable in the lungs of mice that were injected with 5 μg dose (FIG. 15). In both cases, the highest peptide levels were observed 6 hours after the injection. Thus, the pulmonary administered ACE2 protein analogue is stable in the lung, enabling the treatment time to take effect and neutralize the virus (virus present in the lung, newly inhaled virus, and also virus particles being generated inside the lungs, thus slowing down the virus spread).

Analysis of lung tissue using LC-MS/MS based methodology interfaced with nano-chromatography separation looked at the signal generation of both RBD (here the SARS-CoV-2 Spike 51 Receptor Binding Domain Protein) and ACE2-variant within the mouse lung after administration, with data from two consecutive lung tissue measures per time-point. It was found that the two binding partners (ACE2 peptide variant and RBD) move together exactly over time, with high correlation (see FIG. 15 and table 2), they are complexed, as otherwise they would have a high degree of variation. As such, it is shown that the pulmonary administered ACE2 peptide variant interacts with the Coronavirus spike protein (RBD) in vivo.

Also, plasma levels of the peptide were monitored for the mice of the mouse model experiments. ACE2 was detected only in the plasma samples of mice that received 5 μg ACE2, with decreasing concentrations over time. The ACE2 protein was not detectable in mice that received saline or 1 μg ACE2 (FIG. 16). As such, plasma leakage is found minimal.

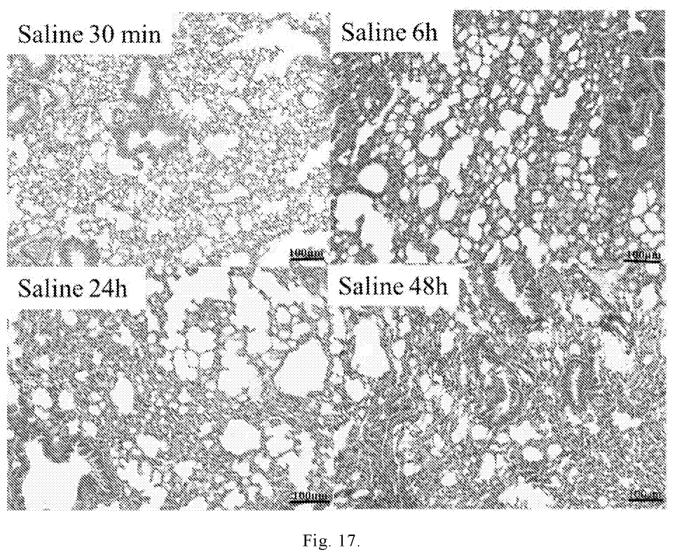

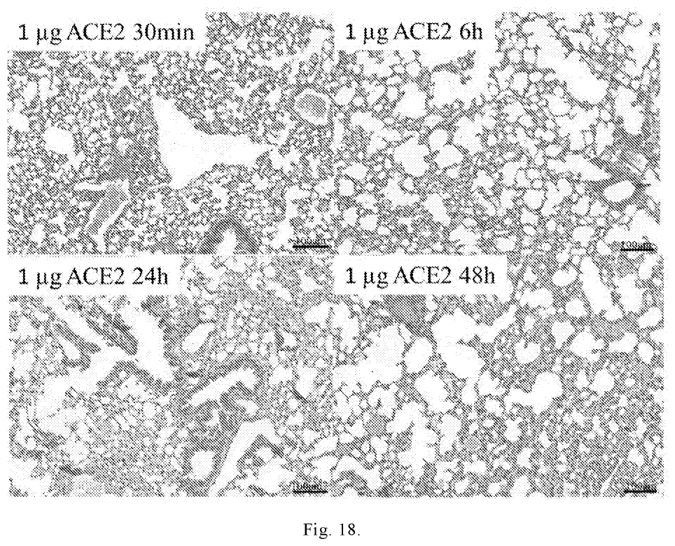

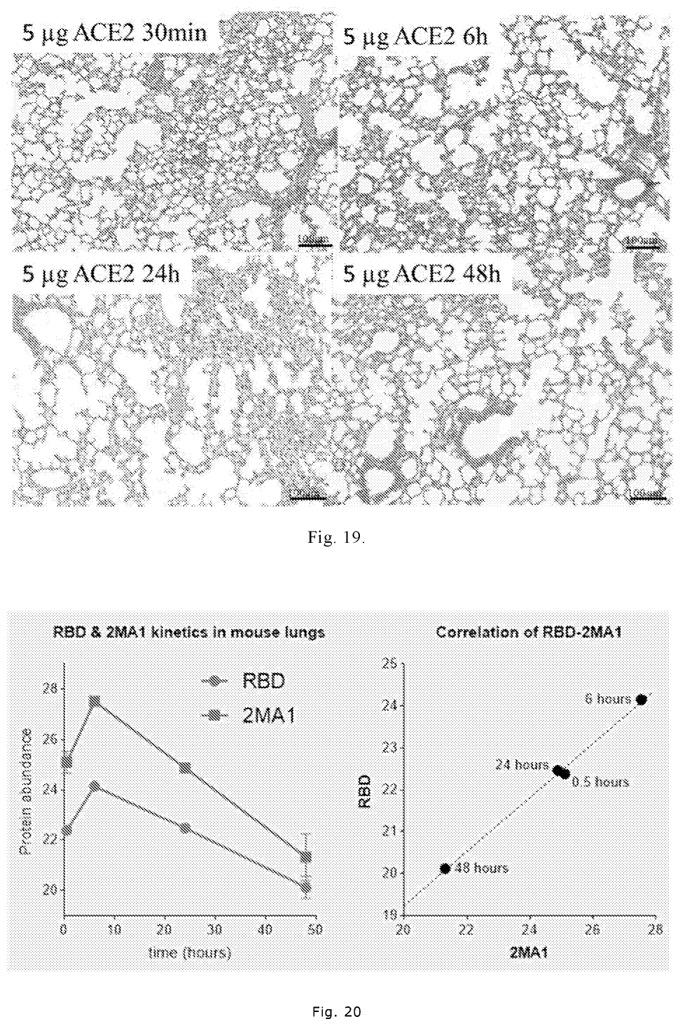

Importantly, microscopic examination of hematoxylin-eosin stainings of lungs did not show any damage or relevant difference in tissue structure between the lungs of mice that received saline, 1 μg or 5 μg ACE2 (FIG. 17-19). This points to that the pulmonary administration of the ACE2 peptide variants does not have negative effect on pulmonary microenvironment and alveolar function.

To confirm the complex formation between 2MA1 and RBD (spike protein) in the lung of the mouse, an experiment was designed to separate the complex form the non-interacting proteins. A filter of 50 kDa will discriminate between the free RBD (˜30 kDa) and the 2MA1-RBD complex (˜120 kDa). The proof of concept was performed with only RBD and the complex 2MA1-RBD pre-incubated. The FIG. 21 shows that free RBD predominantly is collected in the flow-through, while when pre-incubated with 2MA1 is only detected in the supernatant.

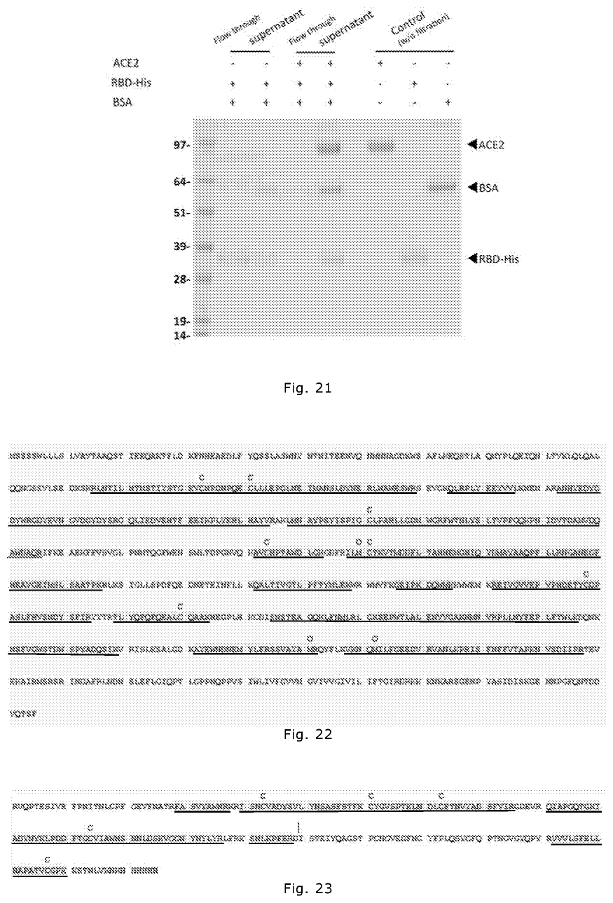

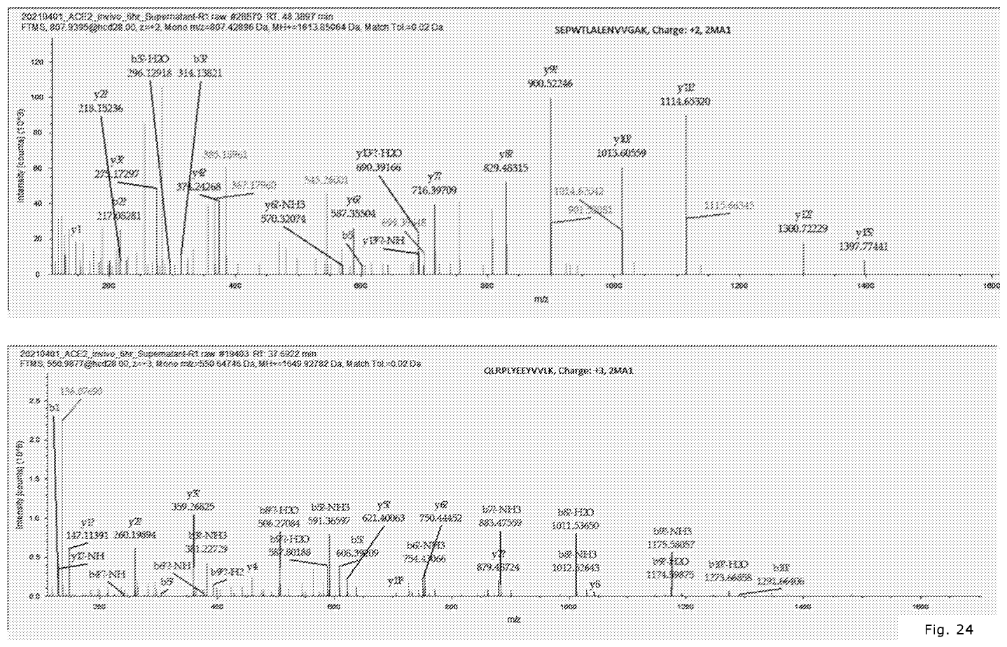

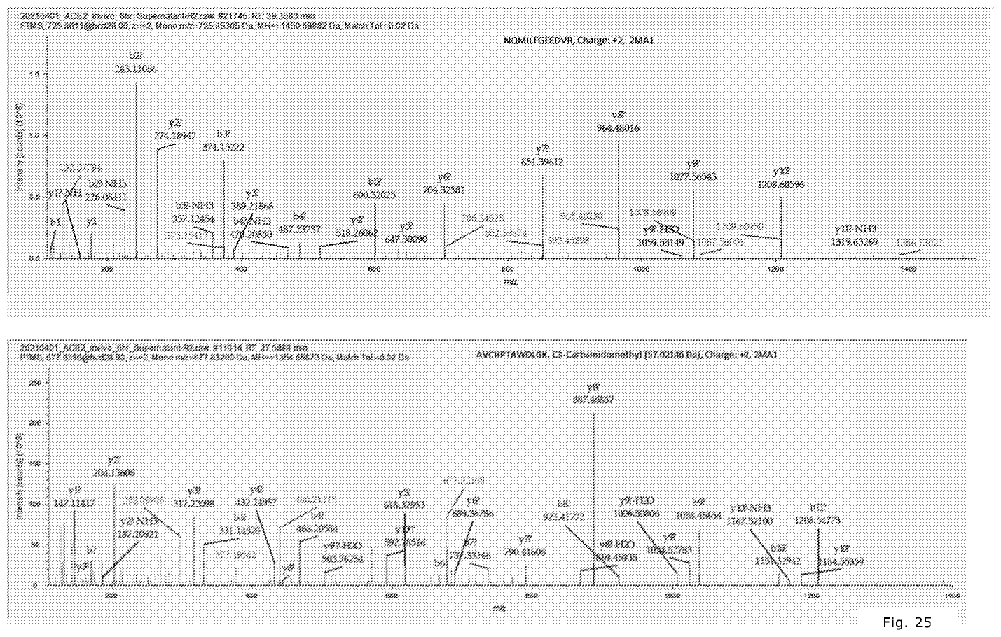

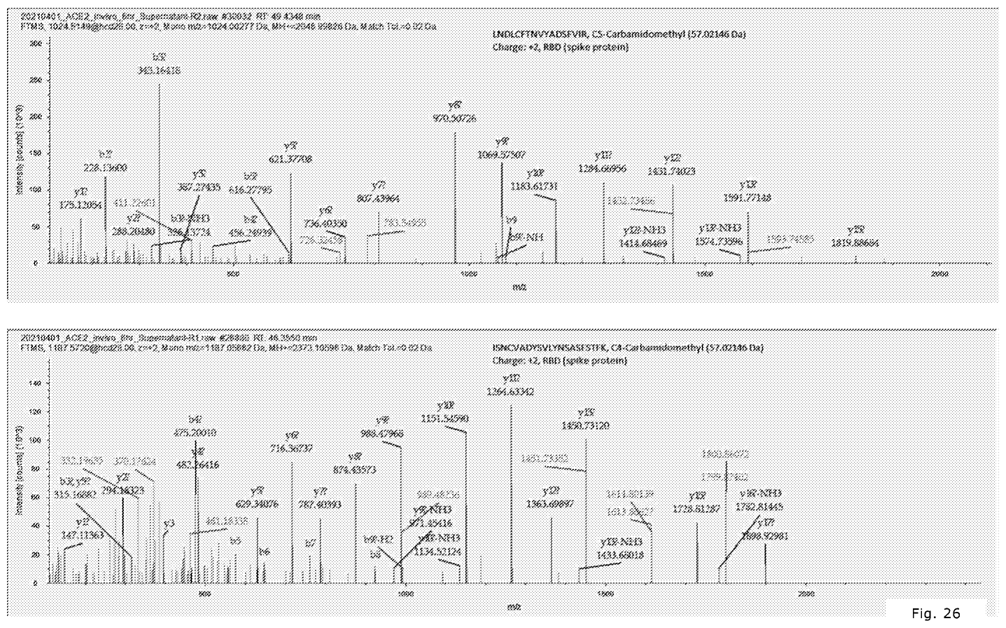

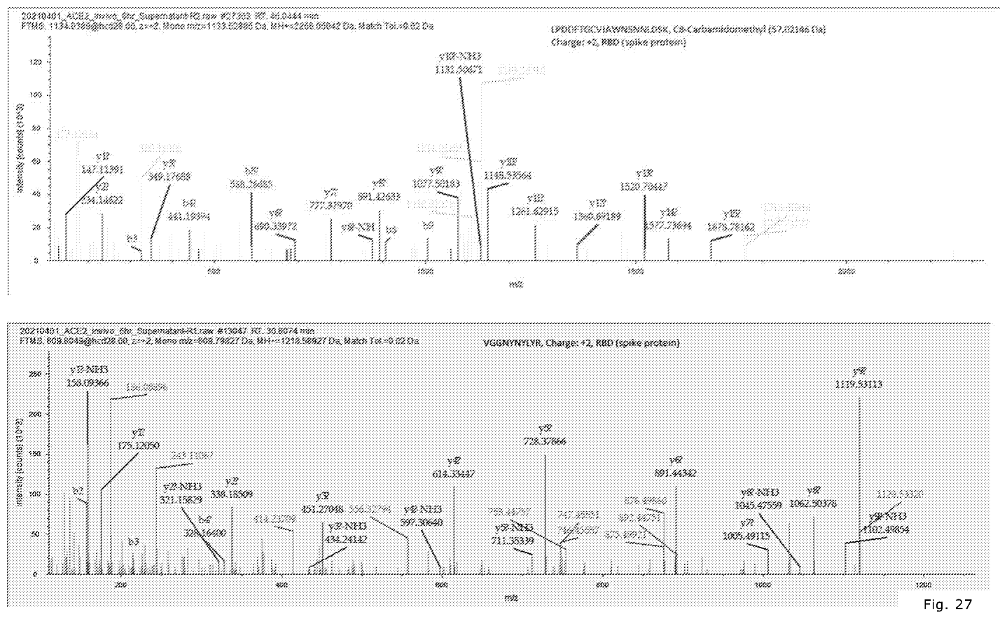

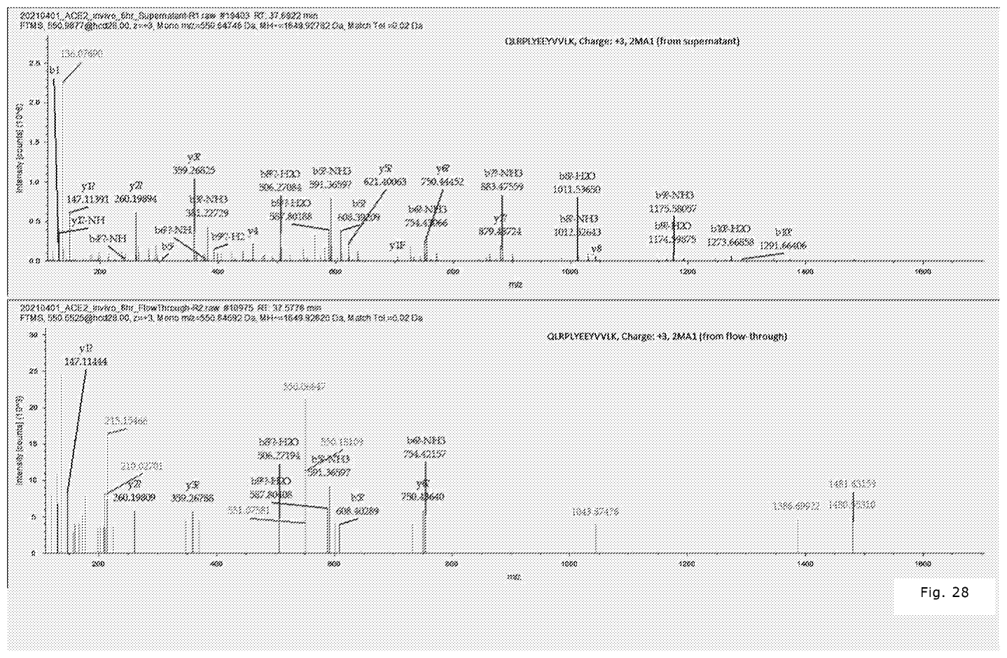

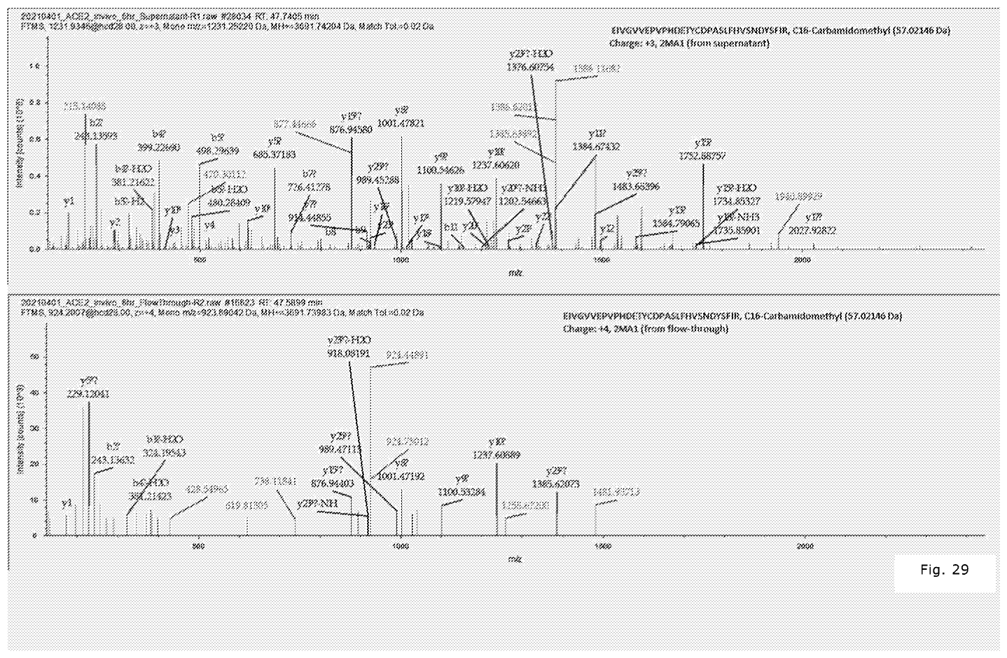

The determination of the complex formation in the lung of mice in vivo was performed at 6 hours after inoculation of mice with a mix of the two proteins (2MA1 and RBD). Proteins from lung tissue were extracted under non-denaturing conditions and submitted to the filtration process. Both the flow-through and the supernatant were immuno-precipitated for 2MA1 and RBD before processing the samples for LC-MS/MS analysis to detect the proteins. The results show that both 2MA1 and RBD were confidently identified in the supernatant. The coverage of the sequences confirmed by mass spectrometry sequencing is underlined (FIG. 22 for 2MA1 and FIG. 23 for RBD). Peptides from both extremes of the proteins were sequenced indicating in addition the integrity of both proteins after 6 hours in the lung of living mice. In total, 27 different peptides from 2MA1 and 10 from RBD were properly sequenced by mass spectrometry, covering more 55% of both of their primary structures. Four representative mass spectra showing the sequencing and the correct assignation to a peptide from the protein were selected for each of the proteins. FIGS. 24 and 25 show four mass spectra correctly assigned to peptide sequences from 2MA1. Similarly, FIGS. 26 and 27 show four spectra and their correct assignation to peptides from RBD protein. The illustrations show how the most intense signals in the spectra are explained by the sequences and assigned to y or b series of ions. The results unequivocally place the two proteins in the supernatant fraction, which strongly suggest that both proteins were in a complex in the lung of mice. The LC-MS/MS analysis of the sample collected in the flow-through did not show any detectable signal from the RBD protein, suggesting that the protein is at very low or absent in its free form in the lung. On the other hand, two weak signals were assigned to two different peptide sequences of 2MA1. The comparisons of the signal intensities and the quality of the spectra between the supernatant and in the flow-through indicate that the amount of 2MA1 detected in the flow-through is below 1% of the amount detected in the supernatant fraction (FIG. 28 and FIG. 29). The top spectra correspond to the supernatant and the bottom to the flow-through (FIGS. 28 and 29). Besides, two replicates of the experiment were performed, and the two signals from 2MA1 were only detected in one of the replicates of the flow-through.

In one embodiment, is provided a dry powder or an aerosol comprising the polypeptide as described above.

In one embodiment, is provided a dry powder or an aerosol comprising at least 75 amino acids and having an amino acid sequence having at least 90%, such as at least 95%, 96%, 97, 98%, or 99%, such as 100% sequence identity (% SI) with SEQ ID NO 1.

In one embodiment, the polypeptide comprises at least 76 amino acids, such as at least 77, 78, 79, 80, 81, 82, 83, or 84 amino acids, and shares at least 90%, such as at least 95%, 96%, 97, 98%, or 99%, such as 100% sequence identity (% SI) with SEQ ID 2 or SEQ ID NO 3.

In one embodiment, the polypeptide comprises at least 400 amino acids, such as at least 405, 406, 407, 408, 409, 410, 411, or 412 amino acids, and shares at least 90%, such as at least 95%, 96%, 97, 98%, or 99%, such as 100% sequence identity (% SI) with SEQ ID 4 or SEQ ID NO 5.

In one embodiment, the polypeptide comprises at least 500 amino acids and having an amino acid sequence having at least 90% sequence identity (% SI) with SEQ ID NO 1.

In one embodiment, the peptide comprises at least 700 amino acids, such as at least 710, 715, 716, 717, 718, 719, 720, 721, or 722 amino acids, and shares at least 90%, such as at least 95%, 96%, 97, 98%, or 99%, such as 100% sequence identity (% SI) with SEQ ID 6 or SEQ ID NO 7.

In embodiment, the polypeptide polypeptide comprises at least 90%, such as at least 95%, 96%, 97, 98%, or 99%, such as 100% sequence identity (% SI) with human ACE2 N-terminal peptidase domain (PD) domain (SEQ ID NO 1), wherein the sequence of the PD domain is amino acids 19-615 of SEQ ID NO 1.

In one embodiment, the peptide shares at least 90%, such as at least 95%, 96%, 97, 98%, or 99%, such as 100% sequence identity (% SI) to the extracellular domain of ACE2, wherein the sequence of the extracellular domain is amino acids 18-740 of SEQ ID NO 1.

In one embodiment, peptide is the proteoform of the human ACE2 gene

In one embodiment, the dry powder or an aerosol is suitable for nasal and/or pulmonary administration.

In one embodiment, the dry powder or an aerosol is suitable for pulmonary administration.

In one embodiment, the dry powder or an aerosol is suitable for nasal administration.

By “pulmonary administration” or “administering pulmonarily” is meant a route of inhalational administration that delivers an effective amount of the compound to the tissues of the lower respiratory tract.

By “nasal administration”, or “administered nasally”, is meant a route of inhalational administration that delivers an effective amount of the compound to the tissues of the nasal tract.

Aerosols can be created in different ways, for instance using nebulizers or vaporisers. Nebulizers use oxygen, compressed air or ultrasonic power to break up the liquid solutions into aerosol (a mix of gas and solid or liquid particles) droplets that can be inhaled directly through a mouthpiece. Vaporizer uses electricity and heating coils to achieve aerosol droplets.

As can be seen in FIGS. 12 and 13, the peptides of the invention works well with different types of dispensers, such as jet and mesh dispensers.

For pulmonary administration, the powder particles or the aerosol droplets are preferably limited to a mass median aerodynamic diameter (MMAD) less than 10 such as less than 9, 8, 7, 6, 5 μm preferably less than 5 μm, or between 0.5 μm and 5 μm. As such, the alveoli of the lung can be effectively targeted for drug absorption by pulmonary delivery.

For powder particles or the aerosol droplets limited to a mass median aerodynamic diameter (MMAD) of 100 to 20 μm, such as 80 to 40 μm, the particles will start to deposit during inhalation, such as in the nasal cavity.

In one embodiment, the particle or droplet size of the dry powder or aerosol is a median aerodynamic diameter (MMAD) of 100 to 0.5 μm, for deposit in the nose and lung.

In one embodiment, the particle or droplet size of the dry powder or aerosol is a median aerodynamic diameter (MMAD) of 100 to 20 μm, such as 80 to 40 μm.

This enables the dry powder or an aerosol to deposit in the nasal tract.

In one embodiment, the particle or droplet size of the dry powder or aerosol is median aerodynamic diameter (MMAD) less than 5 μm.

In one embodiment, the particle or droplet size of the dry powder or aerosol is a median aerodynamic diameter (MMAD) of less than 10 μm, such as less than 9 μm, 8 μm, 7 μm, 6 μm or 5 μm, preferably less than 5 enabling the dry powder or an aerosol to enter the alveoli of the lung.

This enables the dry powder or an aerosol to enter the alveoli of the lung. Inhaled dry powder particles within the preparation should be sized <5 micro meter, at a rate of at least 50% in order for the small sized particles to reach out into the alveolar tract. The dry powder preparation can but is not restricted to contain excipients and other compounds that can have impact on drug absorption, as well as have stabilizing effect on the protein drug.

Preparation of the dry powder peptide or ACE2 formulations may be carried out using a variety of well known methods including lyophilization, spray drying, agglomeration, spray coating, extrusion processes and combinations of these. Preferably, the particle size of the resulting powder is such that more than 95%, preferably more than 98%, of the mass of the dry powder is in particles having a diameter (MMAD) of about 10 μm or less, with more than about 90% of the mass being in particles having a diameter (MMAD) of less than about 5 μm.

In one embodiment, a dry powder formulation of the peptide or ACE2 protein is prepared using drying processes such as agglomeration processes, extrusion, spray coating and lyophilization and jet milling processes.

In one embodiment, a dry powder formulation of the peptide or ACE2 protein is prepared using a spray drying/agglomeration process, which produces a substantially amorphous powder of homogenous constitution having a particle size that is readily respirable.

While it is possible to administer the peptide of the invention in pure form, it is often desirable to include additional components in a pharmaceutical formulation. Such formulations comprise the peptide of the invention in a therapeutically or pharmaceutically effective dose together with one or more pharmaceutically or therapeutically acceptable carriers and optionally other therapeutic ingredients, such as bulking agents, buffers, and other pharmaceutical agents for co-administration. Such additives may provide added characteristics required for a physio-chemical property and effect, such as an improved duration within the lung compartment.

In one embodiment, the powder or an aerosol further comprises bulking agents, buffers, and/or other pharmaceutical agents.

While the dry powder or aerosol of the invention is suitable for pulmonary treatment of COVID-19, it is not limited thereto, and may be used for other disorders, such as for treatment of pulmonary hypertension (PH or PHTN).

TM sprayer (HTXtechnology) was used to generate dry powder of the 2MA1 peptide (SEQ ID NO 2). The small liquid volumes spotted with such devices have low kinetic energy that reduces the risk of protein denaturation. Under the spray nozzle, dry powder from the formulation containing the 2MA1 was collected in a plastic cartridge. The resulting powder was a substantially amorphous powder of homogenous constitution.

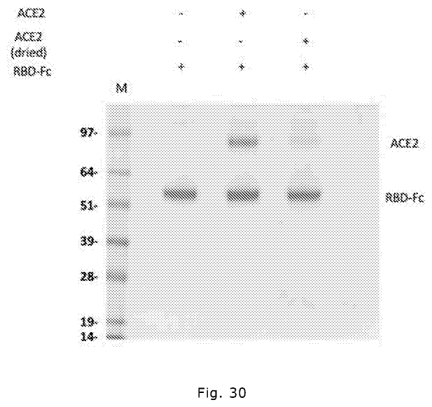

It was found that the powder was stable in room temperature. After 48 hour storage of the dry powder, the powder was reconstituted and characterized. The results clearly indicate the 2MA1 stability during storage and re-solubilization. In FIG. 30, is shown pull-down assay results. It was found that the solubilized dried substance (2MA1 dry powder formulation product) showed similar characteristics as the soluble ACE2 protein band. This confirms that the drying process does not influence on the 2MA1 dry powder formulation product, nor does it affect its binding to RBD.

By “treatment” is meant the full spectrum of therapeutic treatments for a particular disorder ranging from a partial alleviation of symptoms to helping to cure the particular disorder. Treatment is typically effected by the pulmonary administration of a therapeutically effective amount of the peptide of the invention.

By “therapeutically effective amount” is meant an amount of peptide that is sufficient to effect treatment of the particular disorder for which treatment is sought, i.e., sufficient augmentation of peptide levels in the lower respiratory tract.

Typically, treatment of the above described disorders will be affected by administering dosages of the peptide dry powder or aerosol that total in the range of from about 0.1 to about 500 mg, such as 1 mg to 200 mg, such as 20 mg to 50 mg, of peptide or ACE2 daily.

To achieve the desired therapeutic amount, it may be desirable to provide for repeated administrations, i.e., repeated individual inhalations of a metered dose. The individual administrations are repeated until the desired daily dose is administrated, or until satisfactory alleviation of symptoms is provided.

Materials and Methods

ACE2 Extracellular Domain Expression in E coli

Human ACE2 (Met1-Ser740), expressed with a polyhistidine tag at the N- and C-terminus (Host E. coli) was purchased from MP biomedicals (cat #SKU 08720601). This sample is dissolved in 8 M Urea, 20 mM Tris pH8.0, 150 mM NaCl, 200 mM Imidazole, according to the manufacture's document.

Protein Characterization—after 1D Gel Separation and Gel Band Isolation

The samples were diluted in Laemmli buffer and loaded onto the 1D-gel, after the protein separation finished the proteins were stained following a Coomassie brilliant blue (CBB) protocol (alternatively silver staining can be used). A main protein band at 85 kDa consistent 2MA1 (SEQ ID NO 2) was observed and the intensity of bands correlate to amount loaded in the lane. The estimated purity was approximately 95% based on the intensity of all bands detected in the lane, made by stain-intensity determination.

Primary Sequence Confirmation

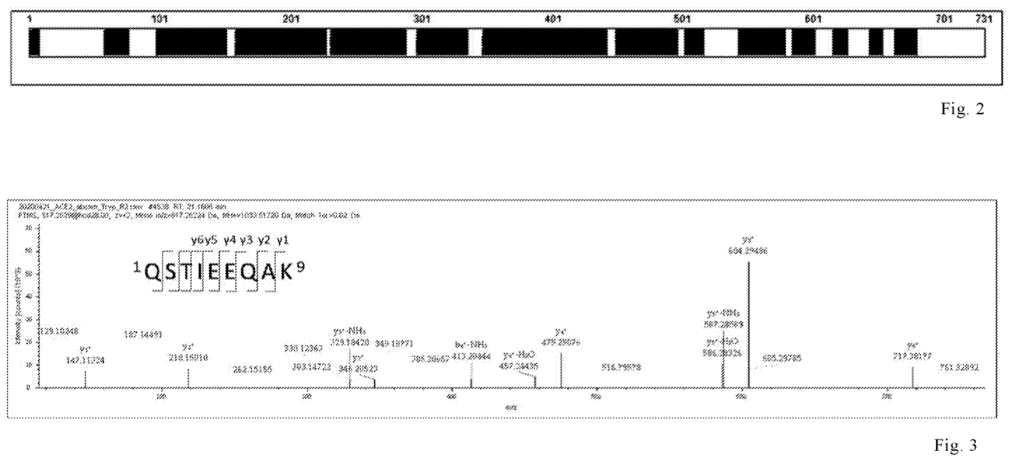

The confirmation of the 2MA1 (SEQ ID NO 2) (95% main-band) primary sequence was performed by high resolution nano-Liquid Chromatography interfaced to high resolution tandem mass spectrometry (MS/MS, a Q Exactive HF-X mass spectrometer coupled to an Ultimate 3000 RSCLnano pump (Thermo Scientific). denamed (LC-MS/MS). The samples were dissolved in ammonium bicarbonate 20 mM, trypsin was added (at a ratio of 1:10, enzyme:substrate relation), and incubated 16 hours at 37° C. The reaction was stopped by adding TFA to a final concentration of 0.1%. The mixture of peptides was next analyzed by LC-MS/MS on an Acclaim PepMap100 C18 (5 μm, 100 Å, 75 μm i.d.×2 cm, nanoViper) chromatography column stationary ohase, was used as trap column and EASY-spray RSLC C18 (2 μm, 100 Å, 75 μm i.d.×25 cm) as analytical column. Solvent A was 0.1% formic acid (FA), solvent B was 80% acetonitrile (ACN) with 0.08% FA. The flow-rate was set to 0.3 μl/min and the column temperature was 45° C. The peptides were separated using a 60 min non-linear gradient and analyzed with a top 20 DDA (data dependent acquisition) method. Generated MS spectra were query to the 2MA1 (SEQ ID NO 2)—Main band theoretical sequence. A total of 35 peptides covering the 71% of 2MA1 (SEQ ID NO 2) amino acid sequence were sequenced. The distribution of the 2MA1 (SEQ ID NO 2) sequence coverage is represented in FIG. 2.

The verification of both extremes of the protein is important for determining the integrity of the molecule. The theoretical N-terminal peptide generated by trypsin digestion is: 1QSTIEEQAK9 with a molecular mass of 1032.51 Da. From the LC-MS/MS analysis a double charged signal at 517.26 Th (1032.51 Da) was fragmented and its MS/MS was correctly assigned to the N-terminal peptide (shown in FIG. 3). The sequencing of the N-terminal peptide confirmed that the molecule preserves its N-terminal as an intact part of the molecule.

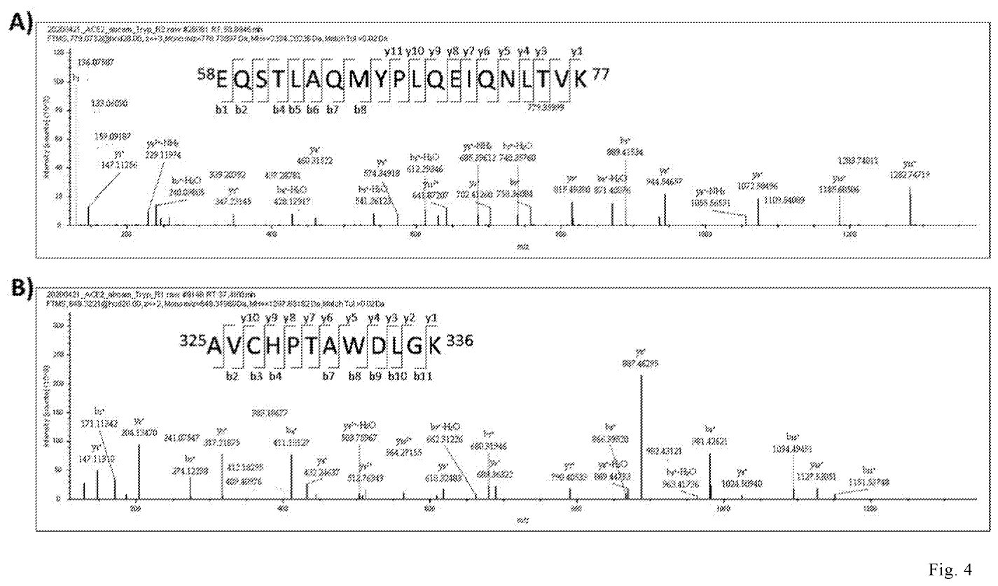

The N-terminal part together with two other regions of the 2MA1 (SEQ ID NO 2)—main band protein is involved in the binding with the spike protein of the virus. One binding site was fully covered by sequencing (FIG. 4A) and the other was sequenced mostly (shown in FIG. 4B).

The protein was cloned and expressed with a His tag in the C-terminal which was used for purification. In addition, we verified that the protein contains its N-terminal intact. The LC-MS/MS analysis allowed us to confirm 71% of the sequence including totally or partially the three binding regions with the spike protein.

Stability Test of 2MA1 (SEQ ID NO 2) by SDS-PAGE Assay

The stability of 2MA1 (SEQ ID NO 2) was investigated with perspective to pH-window and sample composition.

1) SDS-PAGE Assay

As a positive control, 0.6 microgram of 2MA1 (SEQ ID NO 2) in PBS at room temperature was used.

After incubation, the reaction was stopped by adding 5 micro litres of 4×sample buffer (Thermo) and 2.22 micro litres of 0.5 M DTT.

Sample preparation; In order to prepare these samples for protein stability, applying electrophoresis assay, we performed denaturation of 2MA1 (SEQ ID NO 2), sample by heating at 95° C., for 5 min. Next, alkylation was conducted by the addition of 1.78 micro litres of iodo acetoamide (0.5 M).

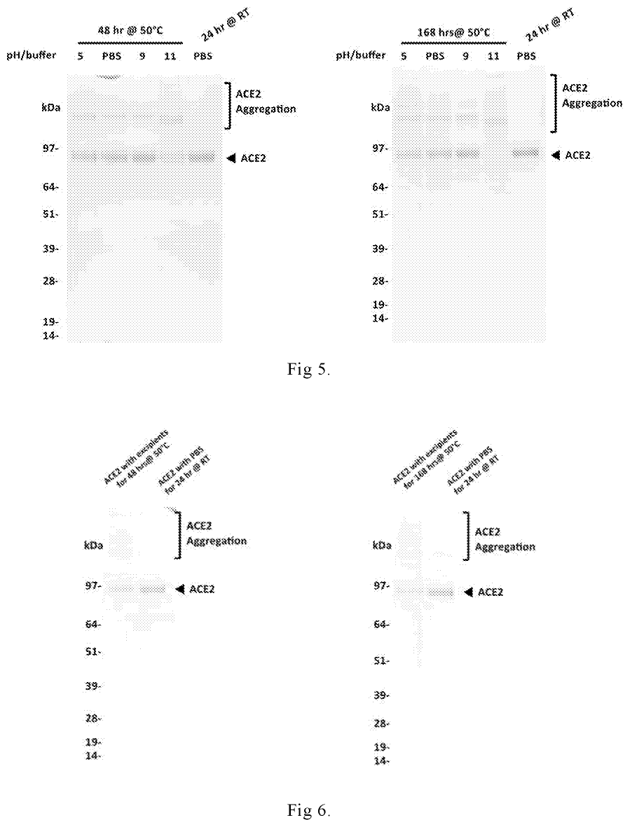

2) pH-Dependence of the Stability (see FIGS. 5 and 6)

Sample preparation of 2MA1 (SEQ ID NO 2)

2MA1 (SEQ ID NO 2) had a concentration of 1.5 mg/mL.

2MA1 (SEQ ID NO 2) was prepared as protein solution, diluted by MilliQ water to 0.6 mg/mL.

0.6 microgram of 2MA1 (SEQ ID NO 2) were incubated in the following buffers or formulation at 50° C. for 24 hrs and 48 hrs;

1) pH 5, 100 mM citrate buffer

2) pH 7.5, PBS.

3) pH 9, 50 mM Tris-HCl

4) pH 11 buffer

3) The stability effect of 2MA1 (SEQ ID NO 2) in formulation

Sample preparation of 2MA1 (SEQ ID NO 2)

2MA1 (SEQ ID NO 2) had a concentration of 1.5 mg/mL.

2MA1 (SEQ ID NO 2) was prepared as protein solution, diluted by MilliQ water to 0.6 mg/mL.

0.6 micrograms of 2MA1 (SEQ ID NO 2) were incubated in the following buffers or formulation at 50° C. for 48 hrs and 168 hrs;

1) 2MA1 (SEQ ID NO 2) in 48 h

2) 2MA1 (SEQ ID NO 2) in 168 h

3) pH 7.5, PBS, 24 h.

Contents of Formulation

Formulation was prepared with the ingredients shown in table 1, with a resulting pH: 7.4, comprising a NaCl concentration of 8.5 mg/ml, Tween 80 concentration of 0.2 mg/ml, Phosphate buffer concentration of 0.7 mg/ml, and EDTA concentration of 0.1 mg/ml.

TABLE 1
Contents of formulation

| Materials | Concentration (mg/mL) |
|---|---|
| NaCl | 8.5 |
| Tween 80 | 0.2 |
| Phosphate buffer | 0.7 |
| EDTA | 0.1 |

SDS-PAGE

All volume of each sample was applied to a well in the gel (NuPAGE 4-12% gel, Thermo). 3 microlitres of Seeblue2 (thermo) was used as a molecular marker. Electrophoresis ran in MOPS buffer system under 200 V for 45 min.

CBB Staining and Gel Scan

Gels were stained by using colloidal blue staining (Thermo) following the manufacturer's instruction. In brief, after fixation of the gels, the gels were staining for 3 hr and de-stained by milliQ water over night. The gels were scanned by HP Scanjet G4050 (HP).

Surface Plasmon Resonance Spectrometry

Formulation stability and properties of 2MA1 (SEQ ID NO 2);

With ACE2, the full extracellular domain 18-740 amino acids, we performed the complex formation assay, identifying the binding properties of 2MA1 (SEQ ID NO 2), with the SPIKE protein of SARS COVID-2. With Surface plasmon resonance (SPR; Biacore platform), where we provide evidence of the interaction with the spike protein, at 25 and 50 nM within the graph below. The concentration dependent signal response for binding is shown in FIG. 7, which means we have the His-tag modified SPIKE protein of SARS COVID-2 bound to the SPR surface, and use the micro-fluidic platform to introduce 2MA1 (SEQ ID NO 2) to the chip surface with the immobilized SPIKE protein. The RBD-Fc was immobilized onto a CM5 micro-fluidic chip at a level of 321.4 Response units (RU). The parallel channel with in the experimental run was the blank, and acted as the reference and background, utilized for the measurements, in order to make normalizations. The 2MA1 (SEQ ID NO 2)—HIS dissociate in 600 seconds.

The binding characteristics between RBD-Fc and different forms of ACE2 (ACE2-His (A-his) and deglycosylated ACE2-His (dA)) were investigated using a BIAcore X-100 instrument (GE Healthcare, Uppsala, Sweden). RBD-Fc was immobilized on a CM5 sensor chip (GE Healthcare) at a level of 321.4 response units using standard amine coupling. In parallel, one flow cell was incubated with buffer alone (i.e. without RBD-Fc), serving as control. Interaction experiments were performed with injections of 15.625, 31.25, 62.5, 125, 250 nM of ACE2-His and deglycosylated ACE2-His in running buffer (0.02 M phosphate buffer with 2.7 mM KCl, 0.137 M NaCl and 0.05% Solubilizer P20 (Tween 20)) at a flow rate of 30 μl/min for 180 seconds. After the end of each injection, dissociation was performed for 600 seconds and then the surface is regenerated with 10 mM Glycine HCl pH 2.5 for 30 seconds, followed by a 10 seconds washing procedure. After X and Y normalization of data, the blank curves from the control flow cell of each injected concentration were subtracted. The BIA evaluation 3.1 analysis software (GE Healthcare) was used to determine equilibrium dissociation dissociation constants (KD) from the processed data sets by fitting to a 1:1 molecular binding model. The binding data can be seen in FIG. 8.

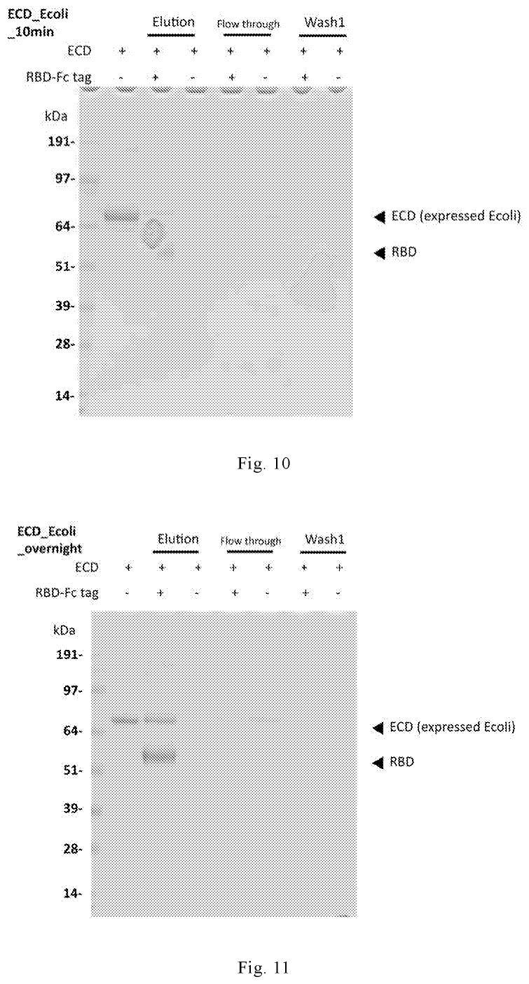

2MA1d—de-glycosylated binding is seen in FIG. 9. In conclusion, 2MA1d (the deglycosylated form of the protein according to SEQ ID NO 2) is weak binding with RBD compared to 2MA1 (SEQ ID NO 2). We run the binding assay for deglycosylated ACE2-His (dA) using 50 and 200 nM of dA as compared to the 25 and 50 nM of ACE2-His (A-His) used on 18th. From this sensogram overlay it was deduced that the binding of dA to RBD-Fc is drastically reduced than the native A-His form.

Intrapulmonary Injection of ACE2 and RBD of the Viral Spike Protein

Soluble rhACE2 (Abcam, Cat. No: ab151852) was dissolved in saline and injected in the lungs of BDF1 mice at two doses (1 and 5 μg protein in 200 μl saline) via tracheostoma. Control animals received only solvent.

The RBD of the viral spike protein (Acro Biosystems, Cat. No: SPD-052H3) and ACE2 were dissolved, mixed in 1:1 molar ratio and injected immediately into the lungs of mice as described above.

Preparation of Tissue Samples

Lungs were harvested 30 min, 6, 24 and 48 hours after the injection and lung lobes were either frozen in liquid nitrogen for Western blot analysis or fixed in formalin and embedded in paraffin for histological analysis.

For collecting plasma from the inner corner of the eye, haematocrit capillaries with sodium-heparin (Deltalab, cat.no. 7401) and 1 ml MiniCollect K3E K3EDTA tubes (Greiner-BioOne, cat.no. 450474) were used. Blood samples of mice (˜200-400 μl) were centrifuged at 1500 rpm for 10 minutes at 4° C., the supernatant was piped into Eppendorf-tubes (˜200 μl), frozen in liquid nitrogen and stored at −80° C. for further investigation.

Western Blot Analysis of Intrapulmonary ACE2 Levels

The lobes of the lungs were homogenized manually with a glass homogenizer in 400 μl Pierce RIPA buffer (Thermo Fisher Scientific, cat.no. 89900) per sample supplemented with 4 μl Protease Inhibitor Cocktail (Sigma-Aldrich, cat.no. P8340), 4 μl 0.5 M EDTA (Thermo Fisher Scientific, cat.no. 15694), 8 μl, 100 mM phenylmethanesulfonyl fluoride in absolute ethanol (Sigma-Aldrich, cat.no. P7626) right before use. Pierce BCA Protein Assay Kit (Thermo Fisher Scientific, cat.no. 23225) was used for determination the protein concentration of the samples in the accordance with the manufacturer's manual. 10 μg protein per sample was loaded for the SDS-polyacrylamide gel electrophoresis. The proteins were electro-transferred onto nitrocellulose membranes. After blotting, the membranes were blocked in 5% BSA/TBS-Tween for one hour. then incubated them with human ACE2 antibody (Invitrogen. cat. no. PAS-110613) in dilution 1:2000 overnight. Blots were then incubated with anti-rabbit (H+L) secondary antibody (Thermo Fisher Scientific. cat.no. 31460) and signals were detected by using SuperSignal West Femto Maximum Sensitivity Substrate (Thermo Fisher Scientific. cat.no. 34095).

Investigation of Plasma ACE2 Levels by Enzyme-Linked Immunosorbent Assay (ELISA)

Human ACE2 ELISA kit was purchased from RayBiotech (cat.no. ELH-ACE2). All plasma samples and kit components were equilibrated to room temperature before the measurement. Sample preparation and detection procedures were performed in the accordance with the manufacturer's manual. The detection range of the assay is 0.025 ng/ml-20 ng/ml. The absorbance was determined at 450 nm with Multiskan Sky microplate reader (Thermo Fisher Scientific. cat.no. 51119600).

Protein Extraction and Enrichment from Tissue Samples

Mouse lung tissue samples were ground on ice was until the tissues were broken. 300 μL of lysis buffer (100 mM Sodium-Phosphate, pH 8.0, 600 mM NaCl, 0.02% Tween-20) was added to the broken tissue, followed by sonication by bioruptor (15 sec ON, 15 sec OFF, 40 cycles. This process ran twice). The sonicated sample was centrifuged at 20,000×g for 3 min @ 4° C.

After centrifugation, supernatant was moved to new 1.5 mL tube. 280 uL of sample was taken out and mixed with 280 μL of milliQ water (to make 1× binding buffer—Use his-tag dynabeads 50 μL (follow the manufacture's instruction), followed by incubation with rotation 5 min in the cold room. Thereafter, it was kept on a magnet for 2 min and supernatant was discarded, followed by 4 washes with 1× binding buffer (300 uL). At the 5th wash, 50 mM phosphate buffer (200 μL) was used and the supernatant was removed.

Digestion

50 uL of 100 mM Ambic was added to the Dynabeads which bind with protein samples, from the protein extraction and enrichment step (above).

1 ug of trypsin (stock solution 1 mg/mL was added and samples were digested for 18.5 hrs at 37° C. @ 500 rpm.

Next day—1.2 uL of 5% TFA (final concentration should be 0.1%) to was added to each sample.

Samples were treated at 95° C. for 5 min @500 rpm, after which samples were spun down. magnet for 2 min to separate the samples from dynabeads and transfer supernatant was transferred to new 1.5 mL tube. This step was repeated to get all sample. Supernatant was speed vac for 1 hr (40-50 min). 20 uL of 0.1% TFA was added to the dried sample to resuspend it, followed by centrifuge at 20,000×g for 3 min. The resulting supernatant was moved to new MS vial for analysis.

Analysis

The identification and quantification of ACE2 (here 2 MAI) was performed by mass spectrometry that is based on a nano-separation chromatography liquid phase separation platform. The separation is interfaced with high-resolution mass spectrometry, utilizing Orbitrap technology. The assay provides quantitative high-resolution, accurate-mass (HRAM) liquid chromatography mass spectrometry (LC-MS) with record-setting performance with the power of built-in software features, which provide elevated sensitivity and selectivity. The Orbitrap technology also delivers depth of analysis to trace levels (attomole level) with high quantitative accuracy and precision.

Sample Preparation; The protein product was dissolved in ammonium bicarbonate 50 mM and digested with trypsin at a 1:10 m:m ratio (enzyme:protein). The enzyme was added and the reaction was incubated for 16 h at 37° C. The reaction was stopped by adding TFA to a final concentration of 0.5%.

Next processing step 1; the generated peptides were analyzed in duplicates by LC-MS. The method of choice MS analysis is usually, but not necessarily Data Dependent Acquisition (DDA) on high-resolution mass spectrometer (HF-X, Thermo). Usually from the MS1 scan the top 20 signal are selected for MS2 fragmentation and excluded for 40 s to be selected again. The normalized collision energy (NCE) is usually fix to 28%. The chromatographic conditions for the separation of peptides usually involve a 1 h non-linear elution gradient for the recommended trap and analytical columns, Acclaim PepMap100 C18 (5 μm, 100 Å, 75 μm i.d.×2 cm, nanoViper) and EASY-spray RSLC C18 (2 μm, 100 Å, 75 μm i.d.×25 cm) respectively.

Next processing step 2; the acquired raw files were submitted to peptide and protein identification. The raw files were processed, but not necessarily with the Proteome Discoverer software (Thermo). The peptides and proteins in the samples were identified by matching the spectra with a human protein database, usually but not necessarily downloaded from UniProt repository. The search engine of choice was usually the Sequest, which was provided together with the Proteome Discoverer. The peptides and proteins identified in the samples were reported using a cutoff for positive identification controlling the FDR at 1%.

Data analysis; Search protein sequence by Proteome Discoverer using fasta file (all mouse sequence and human ACE2, and S-protein sequence)

For the quantification of ACE2 and RBD, the value from “protein abundances” that is simple summation of its associated and used peptide group abundances was used. The value was normalized by log 2 and compared between the samples.

For detection and identification of resulting outcomes, CBB staining and Colloidal Blue Stain kit (Thermo) was used following manufacture's instructions.

Mouse Lung Protein Complex Confirmation Experiments

In order to confirm the complex formation; “ACE2-S Protein”, a sample preparation step was introduced. The ultra-filtration procedure with a 50k Da cut-off (AmiconUltra-0.5 device), was introduced for the recombinant RBD, with and without the ACE2 protein.

To form the “2ACE2-RBD” complex, an incubation was performed of these two proteins for 10 min at room temperature.

In order to prepare the filtration procedure, a preparation was performed, by pre-activation preparation of the filtration device as follows;
Prewash and blocking by BSA 0.1 mg/ml) by the use of 500 μL of milli Q water.
Centrifuge at 14,000×g for 10 min. Discard flow through fraction.
After pre-wash, to block the non-specific binding of proteins, add around 500 μL of BSA (50 μg in MilliQ) sample to the Amicon Ultra filter device and cap it. Place capped filter device into the centrifuge rotor, aligning the caps trap toward the center of the rotor; counter balance with a similar device. Spin the device at 14,000×g for approximately 10 min.

After these steps, the flow through fraction was discarded, where the RBD-His is eliminated (See FIG. 21).

After pre-wash, to block the non-specific binding of proteins, around 500 μL of BSA (50 ug in MilliQ) sample was added to the Amicon Ultra filter device and it was capped. The capped filter device was placed into the centrifuge rotor, aligning the caps trap toward the center of the rotor; counter balance with a similar device. The device was spun at 14,000×g for approximately 10 min.

The flow through fraction was discarded. The filter device was rinsed by pipetting with 500 μL of MilliQ water, which was discarded.

Next, load of sample (RBD or ACE2-RBD complex) to the filtration device was made, followed by centrifugation of the device. After centrifugation, speed vac was used to dry out the samples. Then, add 1×SDS sample buffer and reduce the proteins, and run SDS-PAGE gel separation and stain the gel by CBB (shown in FIG. 21).

The filter Sample preparation procedure worked well in isolating the free RBD-His protein, not being complexed by ACE2. The free RBD go into the flow-through fraction. On the other hand, free RBD didn't go into it. The protein complex stayed in the filtration device.

Sample Processing of RBD-His—ACE2 Protein Complex from Mouse Lung Tissue by Liquid Chromatography-Mass Spectrometry Analysis

To confirm the presence of ACE2-S protein complex within mouse lung tissue, 50k Da cut filtration (AmiconUltra-0.5 device) was used for the lung tissues extracts treated with ACE2-S protein. Two piece of mouse lungs (6 hr treatment) were used. The size of each piece was almost a quarter of the left lung.

Protein extraction was used with 300 uL of lysis buffer (100 mM Sodium-Phosphate, pH 8.0, 600 mM NaCl, 0.02% Tween-20) and Sonicated by bioruptor (15 sec ON, 15 sec OFF, 40 cycles. This process ran twice). After centrifugation at 20,000×g for 3 min @ 4° C.

For the preparation of the filtration device, a prewash was conducted, using 500 μL of milli Q water. Centrifuge at 14,000×g for 10 min. Discard flow through fraction.

After pre-wash, to block the non-specific binding of proteins, around 500 μL of BSA (50 μg in MilliQ) sample was added to the Amicon Ultra filter device and it was capped. The capped filter device was placed into the centrifuge rotor, aligning the caps trap toward the center of the rotor; counter balance with a similar device. Spin the device at 14,000×g for approximately 10 min.

The flow through fraction was discarded, followed by a rinse of the filter device by pipetting with 500 μL of MilliQ water, which was discarded.

The supernatant from the protein extracts was taken to the prepared filtration devices. The device was spun at 14,000×g for approximately 10 min. The flow through fraction was collected to new 1.5 mL tube.

100 μL Milliq water was added to the filter device. To recover the concentrated solute, the Amicon® Ultrafilter device was placed upside down in a clean microcentrifuge tube. It was spun for 2 minutes at 1,000×g to transfer the concentrated sample from the device to the tube. The ultrafiltrate could be stored in the centrifuge tube. Each sample fraction was transferred to 1.5 mL tube, adding equal volume of milliQ water (to adjust the concentration to 1× binding buffer)

For Pull down assay, his-tag dynabeads were used (5 μL) for each flow through and supernatant fraction. Incubation was performed with rotation for 5 min in the cold room. The magnet was kept for 2 min and then the supernatant was discarded. 4 times washing was performed next with 300 μL of 1× binding buffer (50 mM Phosphate buffer pH 8.0, 300 mM NaCl, 0.01% Tween-20). At the 5th wash, 100 mM Ambic (200 μL) was used and the beads-protein complex was transferred to new tube. Supernatants were removed.

Add 50 μL of 100 mM Ambic to the Dyna-beads which bind with protein samples

For reduction the below protocol was followed:
Add 1 uL of 250 mM DTT to sample, and heat 95° C. for 5 min (final conc.: around 5 mM) Use magnet and take supernatant.

To alkylate the proteins, add 1 uL of 250 mM IAA to sample, and for 30 min in dark (final conc.: around 5 mM).

For digestion, add 1 ug of trypsin (stock solution 1 mg/mL)+10 uL of 100 mM Ambic. For digestion during 16 hrs at 37° C. @ 500 rpm:
Add 1.2 μL of 5% TFA (final concentration should be 0.1%) to each sample
Spin down the sample and Speed vac for 1 hr (40-50 min)
Add 20 μL of 0.1% TFA to dried sample and resuspend it
Centrifuge at 20,000×g for 3 min
Take supernatant to new MS vial

Run MS and analyze data by proteome discoverer

MS Data Analysis

MS data analysis showed that 2MA1 and RBD were abundantly identified in the supernatant fraction. The sequence coverage of both proteins were over 55%.

In addition, within the flow through fraction, we were not able to identify the reliable signals of the 2MA1.

The signal response in the flow through fraction was low as compared to the supernatant.

The sequence fragments found are shown in FIGS. 22 and 23, MS spectra for the 2MA1 fragments are shown in FIGS. 24 and 25, and MS spectra for RBD fragments are shown in FIGS. 26 and 27. Comparison spectra between supernatant and filtration are shown in FIGS. 28 and 29.

Dry Powder Formulation

5 μg of ACE2 (Abcam) (2MA1, res 18 to 740) were mixed with 600 μL of Milli Q water. Using a 1 mL of syringe (BD) with syringe pump, the sample was loaded to TM sprayer (HTX technology) at the flow rate of 0.1 mL/min. The sample was sprayed at 28° C. The dispensing system handles volumes down to the volumes of fluid in single cells typically in the 100-200 picoliter region and dispense these with high spatial precision, with a piezodispencing platform. The small liquid volumes spotted with such devices have low kinetic energy that reduces the risk of protein denaturation.

The nozzle of the dispenser ejects small droplets with a high frequency. The dispensing element elongates when a potential is applied leading to compression of the flow-channel and consequently droplet ejection through the nozzle.

Such dispensers, also like piezo-electric ones, deliver highly reproducible liquid volumes, where non-contact spotters, where also solenoid/piezo/ink-jet-based dispensers are being used for the spotting of proteins on microarray surfaces, generating the dry powder.

Dry Powder Reformulation

The dry powder was stored 48 hours in dry conditions at room temperature. With 400 μL of PBS-T (0.02% Tween20), the dried substance was dissolved and collected into a 1.5 mL tube. PBS-T was used as a solvent that is compatible to pull down experiment.

For the pull-down experiment, 3 ug of RBD-Fc tag with dynabeads protein (Thermo) was used. As a control, samples with and without 2 ug of ACE2 was used.

Next, the pull-down assay was run with 1000 uL of solution of dried substance following the manufactures instructions. After performing the pull-down assay, the sample was eluted with 20 μL of the Elution Buffer (50 mM glycine around pH 2.8), and 6.67 μL of premixed NuPAGE LDS 4× Sample Buffer and 2.66 μL of 0.5 M DTT.

After gentle pipetting for resuspension, a heat treatment was performed for 10 minutes at 70° C. Afterwards 20 μL of each sample was applied to the SDS-PAGE gel.

The electrophoresis runs were performed under 150 V constant and MOPS buffer systems.

Results

Stability test of 2MA1 (SEQ ID NO 2) by SDS-PAGE Assay

The Effect on Different pH on 2MA1 (SEQ ID NO 2)

Stability of ACE2 was investigated by different buffer conditions between pH 5 and 11. FIG. 5 demonstrated the pH read-out effects of the stability test. Except for the positive control, gel staining showed extra protein bands upper side of the gel (above 190 kDa). This result indicates that heated ACE2 may have aggregation and/or dimerization. On the other hand, protein form of ACE2 such as degradations were not observed at the lower molecular weight.

In addition, signal intensity of ACE2 at pH 11 was lower than that of others with only approx., 6% after 168 h. According to the European pharmacopoeia 10.0, the pH of liquid preparations of nebulization is not lower than 3 and not higher than 10.

The 2MA1 (SEQ ID NO 2) peptide seems to be stable within the pH window of pH 7-9.

2MA1 (SEQ ID NO 2)—Main Product Characterization

It was verified that the protein contains its N-terminal intact. The LC-MS/MS analysis allowed us to confirm 71% of the sequence including totally or partially the three binding regions with the spike protein.

A total of 35 peptides covering the 71% of 2MA1 (SEQ ID NO 2) amino acid sequence were sequenced. The distribution of the 2 MA sequence coverage is represented in FIG. 2.

The theoretical N-terminal peptide generated by trypsin digestion is: 1QSTIEEQAK9 with a molecular mass of 1032.51 Da. From the LC-MS/MS analysis a double charged signal at 517.26 Th (1032.51 Da) was fragmented and its MS/MS was correctly assigned to the N-terminal peptide (shown in FIG. 3). The sequencing of the N-terminal peptide confirmed that the molecule preserves its N-terminal as an intact part of the molecule.

The N-terminal part together with two other regions of the 2MA1 (SEQ ID NO 2)—main band protein is involved in the binding with the spike protein of the virus. One binding site was fully covered by sequencing (FIG. 4A) and the other was sequenced mostly (shown in FIG. 4B).

Binding of ACE2 Extracellular Domain Expressed in E. coli at 10 Min and 10 h Incubation

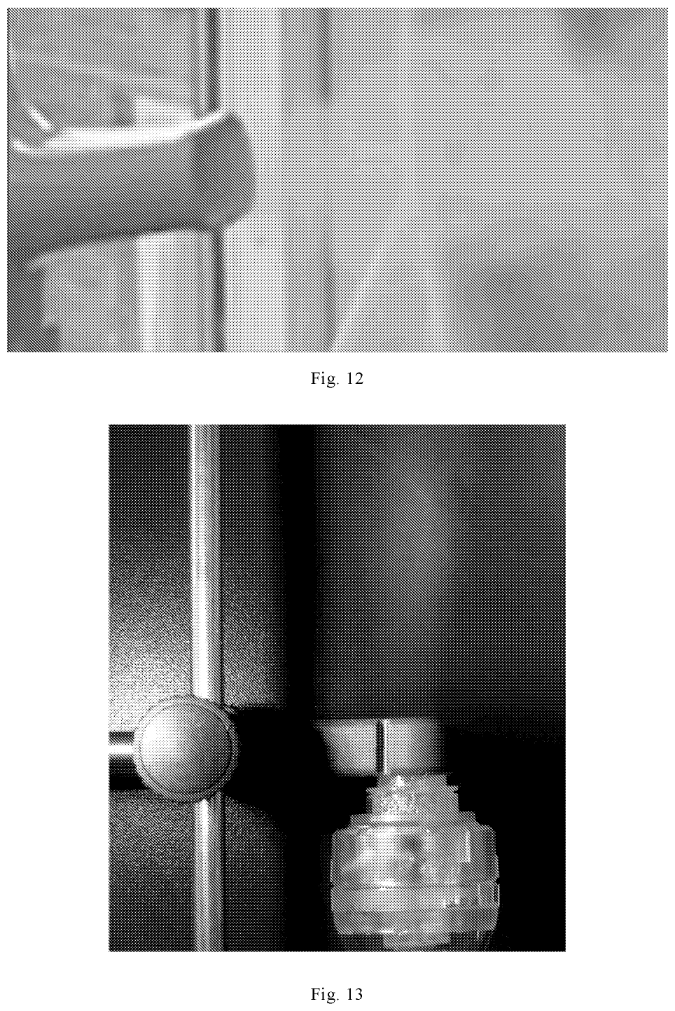

In both 10 min and 10 h incubation, samples (FIGS. 10 and 11), the protein enabled to interact with RBD. In the flow through fraction, the protein in 10 min reaction was detected in flow through fraction. In contrast, protein in overnight reaction sample, wasn't. This result shows that not all of the protein interacted with RBD for 10 min. To overcome numbers of the interacted proteins (ACE2-RBD complex), it is necessary to increase the reaction time.

Intrapulmonary Stability of ACE2 in an In Vivo Mouse Model

The protein was barely detectable in the lungs of mice that received 1 μg ACE2 (FIG. 14), while it was present and very stable in the lungs of mice that were injected with 5 μg dose (FIG. 15). In both cases, the highest protein levels were observed 6 hours after the injection.

Analysis of Circulating ACE2 (2MA1) Levels

We detected ACE2 only in the plasma samples of mice that received 5 μg ACE2, with decreasing concentrations over time. The ACE2 protein was not detectable in mice that received saline or 1 μg ACE2 (FIG. 16).

Histological Evaluation of Lung Tissue Samples

Microscopic examination of hematoxylin-eosin stainings of lungs did not show any damage or relevant difference in tissue structure between the lungs of mice that received saline, 1 μg or 5 μg ACE2 (FIG. 17-19).

Kinetic Profiling of RBD and ACE2-Variant in Mouse Tissue

What we can prove here is that by quantifying the signal generation of both RBD and ACE2-variant within the mouse lung after administration, with data from two lung tissue analyses per time-point.

TABLE 2
Kinetic profiling of RBD and ACE2-variant

| Pearson r | |
|---|---|
| R | 0.9980 |
| 95% confidence interval | 0.9038 to 1.000 |
| R squared | 0.9960 |
| P value | |
| P (two-tailed) | 0.0020 |
| P value summary | ** |
| Significant? (alpha = 0.05) | Yes |
| Number of XY pairs | 4 |

Since the two binding partners move together exactly over time, with high correlation (see FIG. 20 and table 2 above for correlation), they are complexed, as otherwise they would have a high degree of variation.

The assay is a LC-MS/MS based methodology interfaced with nano-chromatography separation.

Mouse Lung Protein Complex Confirmation Experiments

In FIG. 21, the result from the sample preparation step using a 50k Da cut filtration (AmiconUltra-0.5 device) are shown. Isolated free RBD-His protein, not being complexed by ACE2, go into the flow-through fraction (first two columns). On the other hand, the protein complex stayed in the filtration device (second two columns). As such, the 50k Da cut filtration method could be used to probe complex formation in lung tissue.

Sample Processing of RBD-His—ACE2 Protein Complex from Mouse Lung Tissue by Liquid Chromatography-Mass Spectrometry Analysis

To confirm the presence of ACE2-S protein complex within mouse lung tissue, the 50k Da cut filtration was used for the lung tissues extracts treated with ACE2-S protein. The 2 pieces of mouse lungs (from the 6 hr treatment experiments) were used.

MS analysis of the revealed that 2MA1 and RBD were abundantly identified in the supernatant fraction. The sequence coverage of both proteins were over 55%. In total, 27 different peptides from 2MA1 and 10 from RBD were properly sequenced by mass spectrometry. Four representative mass spectra showing the sequencing and the correct assignation to a peptide from the protein were selected for each of the proteins. FIGS. 24 and 25 show four mass spectra correctly assigned to peptide sequences from 2MA1. Similarly, FIGS. 26 and 27 show four spectra and their correct assignation to peptides from RBD protein. The illustrations show how the most intense signals in the spectra are explained by the sequences and assigned to y or b series of ions. The results unequivocally place the two proteins in the supernatant fraction, which strongly suggest that both proteins were in a complex in the lung of mice. In addition, within the flow through fraction, we were not able to identify the reliable signals of the 2MA1. The signal response in the flow through fraction was low as compared to the supernatant. The identified protein fragment sequences are shown in FIGS. 22 and 23. MS spectra for the 2MA1 fragments are shown in FIGS. 24 and 25, and MS spectra for RBD fragments are shown in FIGS. 26 and 27. Comparison spectra between supernatant and filtration are shown in FIGS. 28 and 29. The top spectra correspond to the supernatant and the bottom to the flow-through (FIGS. 28 and 29).

As such, it is clear that the 2MA1 (ACE2) is in complex in the lung tissue samples. Furthermore, the sequence analysis confirms that the complexes are ACE2-RBD complex. Finally, the sample preparation step indicates that the complexes were stable for 6 hours in the lung tissue.

Dry Powder Formulation

Under the spray nozzle, a plastic cartridge was set up, in order to collect the dry powder from the formulation containing the 2MA1. The resulting powder was a substantially amorphous powder of homogenous constitution.

Dry Powder Reformulation

In FIG. 30 is shown the pull-down assay results. The RBD-Fc protein band and control samples showed up in the gel as expected. Thus, negative control was negative and the 2MA1 dry powder formulation product was positive.

The solubilized dried substance (2MA1 dry powder formulation product) was also shown to have the same mobility as the band of the (non-dried) protein in solution. This shows that the drying process does not influence on the 2MA1 dry powder formulation product, and protein structure against RBD.

Although the present invention has been described above with reference to (a) specific embodiment(s), it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

### REFERENCES

1. Zhang H, Penninger J M, Li Y, Zhong N, Slutsky A S. Angiotensin-converting enzyme 2 (ACE2) as a SARS-CoV-2 receptor: molecular mechanisms and potential therapeutic target. Intensive Care Med. 2020; 46(4):586-90. Epub Apr. 3, 2020. doi: 10.1007/s00134-020-05985-9. PubMed PMID: 32125455; PubMed Central PMCID: PMCPMC7079879.
2. Zhou M, Zhang X, Qu J. Coronavirus disease 2019 (COVID-19): a clinical update. Front Med. 2020. Epub Mar. 4, 2020. doi: 10.1007/s11684-020-0767-8. PubMed PMID: 32240462.
3. Coronavirus disease 2019 (COVID-19) [Internet]. UpToDate. 2020 [cited Apr. 7, 2020].
4. Khachfe H H, Chahrour M, Sammouri J, Salhab H, Eldeen B, Mohamad Y. Fares M, et al. An Epidemiological Study on COVID-19: A Rapidly Spreading Disease. Cureus. 2020; 12(3): e7313.
5. Zumla A, Chan J F, Azhar E I, Hui D S, Yuen K Y. Coronaviruses—drug discovery and therapeutic options. Nat Rev Drug Discov. 2016; 15(5):327-47. Epub Feb. 13, 2016. doi: 10.1038/nrd.2015.37. PubMed PMID: 26868298; PubMed Central PMCID: PMCPMC7097181.
6. Hamming I, Timens W, Bulthuis M L, Lely A T, Navis G, van Goor H. Tissue distribution of ACE2 protein, the functional receptor for SARS coronavirus. A first step in understanding SARS pathogenesis. J Pathol. 2004; 203(2):631-7. Epub May 14, 2004. doi: 10.1002/path.1570. PubMed PMID: 15141377.
7. Du L, He Y, Zhou Y, Liu S, Zheng B J, Jiang S. The spike protein of SARS-CoV—a target for vaccine and therapeutic development. Nat Rev Microbiol. 2009; 7(3):226-36. Epub Feb. 10, 2009. doi: 10.1038/nrmicro2090. PubMed PMID: 19198616; PubMed Central PMCID: PMCPMC2750777.
8. Wrapp D, Wang N, Corbett K S, Goldsmith J A, Hsieh C L, Abiona O, et al. Cryo-EM structure of the 2019-nCoV spike in the prefusion conformation. Science. 2020; 367(6483):1260-3. Epub Feb. 23, 2020. doi: 10.1126/science.abb2507. PubMed PMID: 32075877.
9. Hofmann H, Geier M, Marzi A, Krumbiegel M, Peipp M, Fey G H, et al. Susceptibility to SARS coronavirus S protein-driven infection correlates with expression of angiotensin converting enzyme 2 and infection can be blocked by soluble receptor. Biochem Biophys Res Commun. 2004; 319(4):1216-21. Epub Jun. 15, 2004. doi: 10.1016/j.bbrc.2004.05.114. PubMed PMID: 15194496; PubMed Central PMCID: PMCPMC7111153.
10. Li W, Moore M J, Vasilieva N, Sui J, Wong S K, Berne M A, et al. Angiotensin-converting enzyme 2 is a functional receptor for the SARS coronavirus. Nature. 2003; 426(6965):450-4. Epub Dec. 4, 2003. doi: 10.1038/nature02145. PubMed PMID: 14647384; PubMed Central PMCID: PMCPMC7095016.
11. Monteil V, Kwon H, Prado P, Hagelkrüys A, Wimmer R A, Stahl M, et al. Inhibition of SARS-CoV-2 infections in engineered human tissues using clinical-grade soluble human ACE2. Cell. 2020. doi: 10.1016/j.cell.2020.04.004.
12. A G A B. APEIRON Biologics Initiates Phase II Clinical Trial of APN01 for Treatment of COVID-19. 2020.
13. Vaduganathan M, Vardeny O, Michel T, McMurray J J V, Pfeffer M A, Solomon S D. Renin-Angiotensin-Aldosterone System Inhibitors in Patients with Covid-19. N Engl J Med. 2020. Epub Apr. 1, 2020. doi: 10.1056/NEJMsr2005760. PubMed PMID: 32227760.
14. Wolfel R, Corman V M, Guggemos W, Seilmaier M, Zange S, Muller M A, et al. Virological assessment of hospitalized patients with COVID-2019. Nature. 2020. Epub Apr. 3, 2020. doi: 10.1038/s41586-020-2196-x. PubMed PMID: 32235945.
15. Yan R, Zhang Y, Li Y, Xia L, Guo Y, Zhou Q. Structural basis for the recognition of SARS-CoV-2 by full-length human ACE2. Science. 2020; 367(6485):1444-8. Epub Mar. 3, 2020. doi: 10.1126/science.abb2762. PubMed PMID: 32132184.
16. Li W, Zhang C, Sui J, Kuhn J H, Moore M J, Luo S, et al. Receptor and viral determinants of SARS-coronavirus adaptation to human ACE2. EMBO J. 2005; 24(8):1634-43. Epub Mar. 26, 2005. doi: 10.1038/sj.emboj.7600640. PubMed PMID: 15791205; PubMed Central PMCID: PMCPMC1142572.
17. Stawiski E W, Diwanji D, Suryamohan K, Gupta R, Fellouse F A, Sathirapongsasuti J F, et al. Human ACE2 receptor polymorphisms predict SARS-CoV-2 susceptibility. bioRxiv. 2020:2020.04.07.024752. doi: 10.1101/2020.04.07.024752.
18. Myers E W, Miller W. Optimal alignments in linear space. Bioinformatics. 1988; 4(1):11-7. doi: 10.1093/bioinformatics/4.1.11.
19. Pearson W R. Rapid and sensitive sequence comparison with FASTP and FASTA. Methods Enzymol. 1990; 183:63-98. Epub Jan. 1, 1990. doi: 10.1016/0076-6879(90)83007-v. PubMed PMID: 2156132.
20. Altschul S F, Madden T L, Schaffer A A, Zhang J, Zhang Z, Miller W, et al. Gapped BLAST and PSI-BLAST: a new generation of protein database search programs. Nucleic Acids Res. 1997; 25(17):3389-402. Epub Sep. 1, 1997. doi: 10.1093/nar/25.17.3389. PubMed PMID: 9254694; PubMed Central PMCID: PMCPMC146917.
21. Devereux J, Haeberli P, Smithies O. A comprehensive set of sequence analysis programs for the VAX. Nucleic Acids Res. 1984; 12(1 Pt 1):387-95. Epub Jan. 11, 1984. doi: 10.1093/nar/12.1part1.387. PubMed PMID: 6546423; PubMed Central PMCID: PMCPMC321012.
22. Kuba K, Imai Y, Penninger J M. Multiple functions of angiotensin-converting enzyme 2 and its relevance in cardiovascular diseases. Circ J. 2013; 77(2):301-8. Epub Jan. 19, 2013. doi: 10.1253/circj.cj-12-1544. PubMed PMID: 23328447.
23. Dagenais N J, Jamali F. Protective effects of angiotensin II interruption: evidence for antiinflammatory actions. Pharmacotherapy. 2005; 25(9):1213-29. Epub Sep. 17, 2005. doi: 10.1592/phco.2005.25.9.1213. PubMed PMID: 16164395.
24. Rajagopalan S, Kurz S, Munzel T, Tarpey M, Freeman B A, Griendling K K, et al. Angiotensin II-mediated hypertension in the rat increases vascular superoxide production via membrane NADH/NADPH oxidase activation. Contribution to alterations of vasomotor tone. J Clin Invest. 1996; 97(8):1916-23. Epub Apr. 15, 1996. doi: 10.1172/JCI118623. PubMed PMID: 8621776; PubMed Central PMCID: PMCPMC507261.
25. Phillips M I, Kagiyama S. Angiotensin II as a pro-inflammatory mediator. Curr Opin Investig Drugs. 2002; 3(4):569-77. Epub Jul. 2, 2002. PubMed PMID: 12090726.
26. Celec P. Nuclear factor kappa B—molecular biomedicine: the next generation. Biomed Pharmacother. 2004; 58(6-7):365-71. Epub Jul. 24, 2004. doi: 10.1016/j.biopha.2003.12.015. PubMed PMID: 15271418.
27. Boehm M, Nabel E G. Angiotensin-converting enzyme 2—a new cardiac regulator. N Engl J Med. 2002; 347(22):1795-7. Epub Nov. 29, 2002. doi: 10.1056/NEJMcibr022472. PubMed PMID: 12456857.
28. Gironacci M M, Coba M P, Pena C. Angiotensin-(1-7) binds at the type 1 angiotensin II receptors in rat renal cortex. Regul Pept. 1999; 84(1-3):51-4. Epub Oct. 27, 1999. doi: 10.1016/s0167-0115(99)00067-1. PubMed PMID: 10535408.
29. Passos-Silva D G, Verano-Braga T, Santos R A. Angiotensin-(1-7): beyond the cardio-renal actions. Clin Sci (Lond). 2013; 124(7):443-56. Epub Dec. 20, 2012. doi: 10.1042/CS20120461. PubMed PMID: 23249272.
30. Bader M, Ganten D. Update on tissue renin-angiotensin systems. J Mol Med (Berl). 2008; 86(6):615-21. Epub Apr. 17, 2008. doi: 10.1007/s00109-008-0336-0. PubMed PMID: 18414822.
31. Imai Y, Kuba K, Rao S, Huan Y, Guo F, Guan B, et al. Angiotensin-converting enzyme 2 protects from severe acute lung failure. Nature. 2005; 436(7047):112-6. Epub Jul. 8, 2005. doi: 10.1038/nature03712. PubMed PMID: 16001071; PubMed Central PMCID: PMCPMC7094998.

1. A method of treatment of COVID19, SARS, or MERS, comprising:
administering a polypeptide comprising at least 75 amino acids and having an amino acid sequence having at least 90%, such as at least 95%, 96%, 97%, 98%, or 99%, such as 100% sequence identity (% SI) with human Angiotensin-converting enzyme 2 (ACE2) (SEQ ID NO: 1), to a patient in need thereof by a pulmonary and/or a nasal route of administration.

2. The method according to claim 1, for use in the treatment of COVID19.

3. The method according to claim 1, wherein the polypeptide comprises at least 76 amino acids, such as at least 77, 78, 79, 80, 81, 82, 83, or 84 amino acids, and shares at least 90%, such as at least 95%, 96%, 97%, 98%, or 99%, such as 100% sequence identity (% SI) with SEQ ID NO: 2 or SEQ ID NO: 3.

4. The method according to claim 1, wherein the polypeptide comprises at least 400 amino acids, such as at least 405, 406, 407, 408, 409, 410, 411, or 412 amino acids, and shares at least 90%, such as at least 95%, 96%, 97%, 98%, or 99%, such as 100% sequence identity (% SI) with SEQ ID NO: 4 or SEQ ID NO: 5.

5. The method according to claim 1, wherein the polypeptide comprises at least 500 amino acids and having an amino acid sequence having at least 90% sequence identity (% SI) with human Angiotensin-converting enzyme 2 (SEQ ID NO: 1).

6. The method according to claim 1, wherein the polypeptide comprises at least 700 amino acids, such as at least 710, 715, 716, 717, 718, 719, 720, 721, or 722 amino acids, and shares at least 90%, such as at least 95%, 96%, 97%, 98%, or 99%, such as 100% sequence identity (% SI) with SEQ ID NO: 6 or SEQ ID NO: 7.

7. The method according to claim 1, wherein the polypeptide shares at least 90%, such as at least 95%, 96%, 97%, 98%, or 99%, such as 100% sequence identity (% SI) with the extracellular domain of human ACE2.

8. The method according to claim 1, wherein the polypeptide shares at least 90%, such as at least 95%, 96%, 97%, 98%, or 99%, such as 100% sequence identity (% SI) with ACE2 N-terminal peptidase (PD) domain.

9. The method according to claim 8, wherein the sequence of the ACE2 PD domain is amino acids 19 to 615 of SEQ ID NO: 1.

10. The method according to claim 1, wherein the polypeptide is the proteoform of the human ACE2 gene.

11. The method according to claim 1, wherein the polypeptide has anti-inflammatory effect through ACE2 activity.

12. The method according to claim 1, wherein the polypeptide is co-administered with an anti-inflammatory agent and/or an immunosuppressant.

13. The method according to claim 11, wherein the patient has more advanced stage of the infection or severe symptoms, such as acute respiratory distress syndrome (ARDS).

14. A method of treatment of SARS-CoV-2, SARS-CoV, or Mers-CoV in a subject infected with SARS-CoV-2, SARS-CoV, or Mers-CoV comprising:
administration of a polypeptide comprising at least 75 amino acids and having an amino acid sequence with at least 90%, such as at least 95%, 96%, 97%, 98%, or 99%, such as 100% sequence identity (% SI) with human ACE2 (SEQ ID NO: 1) wherein a number of active virus particles being exhaled by the subject infected by SARS-CoV-2, SARS-CoV, or Mers-CoV is reduced following the administration.

15. The method according to claim 14, wherein the subject is infected by SARS-CoV-2.

16. The method according to claim 14, wherein the polypeptide comprises at least 76 amino acids, such as at least 77, 78, 79, 80, 81, 82, 83, or 84 amino acids, and shares at least 90%, such as at least 95%, 96%, 97%, 98%, or 99%, such as 100% sequence identity (% SI) with SEQ ID NO: 2 or SEQ ID NO: 3.

17. The method according to claim 14, wherein the polypeptide comprises at least 400 amino acids, such as at least 405, 406, 407, 408, 409, 410, 411, or 412 amino acids, and shares at least 90%, such as at least 95%, 96%, 97%, 98%, or 99%, such as 100% sequence identity (% SI) with SEQ ID NO: 4 or SEQ ID NO: 5.

18. The method according to claim 14, comprising at least 500 amino acids and having an amino acid sequence with at least 90% sequence identity (% SI) with human ACE2 (SEQ ID NO: 1).

19. The method according to claim 14, wherein the polypeptide comprises at least 700 amino acids, such as at least 710, 715, 716, 717, 718, 719, 720, 721, or 722 amino acids, and shares at least 90%, such as at least 95%, 96%, 97%, 98%, or 99%, such as 100% sequence identity (% SI) with SEQ ID NO: 6 or SEQ ID NO: 7.

20. The method according to claim 14, wherein the polypeptide comprises at least 90%, such as at least 95%, 96%, 97%, 98%, or 99%, such as 100% sequence identity (% SI) with human ACE2 N-terminal peptidase domain (PD) domain (SEQ ID NO: 1), wherein the sequence of the PD domain is amino acids 19-615 of SEQ ID NO: 1.

21. The method according to claim 14, wherein the peptide shares at least 90%, such as at least 95%, 96%, 97%, 98%, or 99%, such as 100% sequence identity (% SI) to the extracellular domain of ACE2, wherein the sequence of the extracellular domain is amino acids 18-740 of SEQ ID NO: 1.

22. A dry powder or an aerosol comprising a polypeptide comprising at least 75 amino acids and having an amino acid sequence having at least 90%, such as at least 95%, 96%, 97%, 98%, or 99%, such as 100% sequence identity (% SI) with human Angiotensin-converting enzyme 2 (SEQ ID NO: 1).

23. The dry powder or aerosol according to claim 22, wherein the dry powder or an aerosol is suitable for pulmonary and/or nasal administration.

24. The dry powder or aerosol according to claim 22, wherein the polypeptide comprises at least 76 amino acids, such as at least 77, 78, 79, 80, 81, 82, 83, or 84 amino acids, and shares at least 90%, such as at least 95%, 96%, 97%, 98%, or 99%, such as 100% sequence identity (% SI) with SEQ ID NO: 2 or SEQ ID NO: 3.

25. The dry powder or aerosol according to claim 22, wherein the polypeptide comprises at least 400 amino acids, such as at least 405, 406, 407, 408, 409, 410, 411, or 412 amino acids, and shares at least 90%, such as at least 95%, 96%, 97%, 98%, or 99%, such as 100% sequence identity (% SI) with SEQ ID NO: 4 or SEQ ID NO: 5.

26. The dry powder or aerosol according to claim 22, wherein the polypeptide comprises at least 500 amino acids and having an amino acid sequence having at least 90%, such as at least 95%, sequence identity (% SI) with human Angiotensin-converting enzyme 2 (SEQ ID NO: 1).

27. The dry powder or aerosol according to claim 22, wherein the polypeptide comprises at least 700 amino acids, such as at least 710, 715, 716, 717, 718, 719, 720, 721, or 722 amino acids, and shares at least 90%, such as at least 95%, 96%, 97%, 98%, or 99%, such as 100% sequence identity (% SI) with SEQ ID NO: 6 or SEQ ID NO: 7.

28. The dry powder or aerosol according to claim 22, wherein the polypeptide comprises at least 90%, such as at least 95%, 96%, 97%, 98%, or 99%, such as 100% sequence identity (% SI) with human ACE2 N-terminal peptidase domain (PD) domain (SEQ ID NO: 1), wherein the sequence of the PD domain is amino acids 19-615 of SEQ ID NO: 1.

29. The dry powder or aerosol according to claim 22, wherein the peptide shares at least 90%, such as at least 95%, 96%, 97%, 98%, or 99%, such as 100% sequence identity (% SI) to the extracellular domain of ACE2, wherein the sequence of the extracellular domain is amino acids 18-740 of SEQ ID NO: 1.

30. The dry powder or aerosol according to claim 22, wherein the polypeptide is the proteoform of the human ACE2 gene.

31. The dry powder or aerosol according to claim 22, wherein the particle or droplet size of the dry powder or aerosol is a median aerodynamic diameter (MMAD) of less than 10 μm, such as less than 9 μm, 8 μm, 7 μm, 6 μm or 5 μm, preferably less than 5 μm, enabling the dry powder or an aerosol to enter the alveoli of the lung.

32. The dry powder or an aerosol according to claim 22, further comprising one or several bulking agents, buffers, and/or other pharmaceutical agents.


SEQUENCE LISTING (7 sequences)

SEQ ID NO: 1 - protein, 805 aa, Homo sapiens [sequence omitted]

SEQ ID NO: 2 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 723 aa, Homo sapiens
QSTIEEQAKTFLDKFNHEAEDLFYQSSLASWNYNTNITEENVQNMNNAGDKWSAFLKEQSTLAQMYPLQEIQDAKIKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 463 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEM

SEQ ID NO: 5 - protein, 463 aa, Homo sapiens
QSTIEEQAKTFLDKFNHEAEDLFYQSSLASWNYNTNITEENVQNMNNAGDKWSAFLKEQSTLAQMYPLQEIQDAKIKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEM

SEQ ID NO: 6 - protein, 86 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQN

SEQ ID NO: 7 - protein, 86 aa, Homo sapiens
QSTIEEQAKTFLDKFNHEAEDLFYQSSLASWNYNTNITEENVQNMNNAGDKWSAFLKEQSTLAQMYPLQEIQDAKIKLQLQALQQN